(19) United States
(12) Patent Application Publication
(10) Pub. No.: US 2004/0034288 A1
(43) Pub. Date: Feb. 19, 2004

(54) Chronic disease outcomes education and communication system

(76) Inventors: Gary R. Hennessy, Bloomfield, CT (US); Ronald F. Larity, Windsor, CT (US)

Correspondence Address: EDWARDS & ANGELL, LLP, P.O. BOX 9169, BOSTON, MA, 02209, US

(21) Appl. No.: 10/468,201

(22) PCT Filed: Feb. 20, 2002
(86) PCT No.: PCT/US02/05040

(51) Int. Cl. A61B 5/00

(52) U.S. Cl. 600/300

(57) ABSTRACT

A chronic disease outcomes, communication, and education system used in connection with a patient is disclosed. The system comprises a database for storing a plurality of patient data entries, each of the patient data entries including personal information of a patient and a guideline concerning the patient's care, the guideline including a default test value associated with monitoring the chronic disease, a user interface for entering patient entries for storage in the database and queries related to patient monitoring, communication and education, and a processor for retrieving the patient data entries from the database in response to the queries from the user interface and storing the patient data entries in the database. The processor provides patient and population outcomes analysis according to an algorithm.

### CROSS REFERENCE TO RELATED APPLICATIONS

Priority is claimed from provisional application Serial No. 60/270,329, filed Feb. 21, 2001.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF THE INVENTION

This invention relates to data processing systems, and more particularly, to an integrated system and method for managing chronic disease that allows patient outcomes analyses, communications, and education.

### BACKGROUND OF THE INVENTION

The concept of cost containment and efficiency of medical care services, commonly known as managed care, has taken on significant importance in the health care industry. Pay providers, in the form of employers, government agencies, insurance companies, health care maintenance organizations, and the like, frequently set forth a series of thresholds which must be established before a patient may have covered access to medical services. Communication of the patient's etiology, treatment plan and updating any changes thereto, is tremendously cumbersome, requiring countless hours by medical providers and their staff to insure this information is organized and accurately communicated to the pay provider, as well as the patient, so that the patient may access covered services and optimize treatment. Further, it is often difficult for the medical provider and/or pay provider to measure the success of the services rendered to the patient and/or the patient's own follow up with the treatment plan.

Certain chronic diseases, such as diabetes, have known etiologies and associated risk factors. Guidelines for treatment have been promulgated by, e.g. the American Diabetes Association, the National Commission for Quality Assurance (NCQA) and Diabetes Quality Improvement Project (DQUIP). These guidelines incorporate known complications associated with diabetes such as retinopathy, neuropathy, nephropathy, Pulmonary Vascular Disease (PVD), Cardial Artery Disease (CAD), and cerebral vascular disease. In addition to various tests associated with monitoring the diabetes, such as HbAlc (measuring glycosolated hemoglobin levels), microalbumin (blood protein), lipids (cholesterol), etc., the physician must typically perform routine eye and foot examinations to monitor the progress of the disease. These tests are in conjunction with those examinations normally associated with an office visit, i.e. blood pressure, temperature, weight, pulse, etc. In addition, there is a significant education and behavior component to the treatment of the disease which can encompass such items as nutrition counseling, smoking cessation, and self education about the disease. The Center for Disease Control estimates that diabetes is reaching epidemic proportions in the United States. Effective treatment centers on the known parameters and risk factors associated with the disease, and insuring that the patient is meeting the objectives of the treatment plan.

The patient's ability to self-monitor blood glucose values at home has significantly improved the ability of the patient (and medical provider) to control the progress of the disease. Hand held monitoring units, such as disclosed in U.S. Pat. No. 4,731,726 to Allen, III, allows the patient to have a portable monitor which generates test values for the blood glucose level and stores the test results. The data may then be downloaded and/or transferred to a computer. The monitor may generate a recommendation to the patient based on patient data, physician input data, and test results, such as an increased insulin dosage. U.S. Pat. No. 5,251,126 to Kahn et al illustrates another diabetes data analysis and interpretation method that identifies insulin intake regimens and identifies statistically significant changes in blood glucose levels in relationship to the insulin levels.

The use of computers to generate a patient record registry and to record data associated with the treatment of those patients enhances the provider's ability to assess the patient's health and generate an assessment plan. U.S. Pat. No. 5,262,943 to Thibado et al discloses a system that receives standardized test data as well as a therapists's subjective evaluations to generate an assessment report for the care of an individual in the mental health field. U.S. Pat. No. 5,265,010 to Evans-Paginelli discloses a hospital patient document method and apparatus that is used to generate an initial patient health care plan, identifying the patient's problems, expected outcomes and interventions to achieve those outcomes.

The use of statistical analysis to create a diagnostic model for a given disease has been employed to create trained neural networks. U.S. Pat. No. 5,769,074 to Barnhill et al, discloses a computer based method which employs the steps of collecting data about patients (such as biological, physical, demographic, racial, environmental); digitizing the data and medical historical data; selecting digitized values that are associated with the diagnosis of a disease; scaling the data; performing tests to analyze the discriminating power of the data; grouping individual data values; preprocessing the data; inputting selected data to make pre-processed values into a computer based neural network in order to train the neural network; analyzing the contributions of the individual data inputs to the network; selecting the optimally trained neural network based on the performance, accuracy and cost; and inputting other patient data into the neural network to produce an output value which indicates whether the patient may have or be susceptible to the disease. Such technology has application to diagnostic patterns that are too subtle or too complex for humans and conventional computational methods to identify and allow for the provider to access large neural networks that are capable of recognizing diagnostic patterns. U.S. Pat. No. 5,860,917 to Comanor, et al, discloses such a neural network with a statistical model derived using a robustified similarity metrical least squares (SMILES) analysis.

In contrast to the neural network developed through statistical analysis of patient data and risk factors to create a diagnostic protocol, certain chronic diseases, such as diabetes, have a known and highly defined treatment protocol. Though incurable, the risk factors associated with diabetes and the complications of diabetes have been well studied. The diabetic patient, however, must be closely monitored to control the disease. It is estimated, however, that physicians associated with the treatment of diabetes do not use computer based data systems to manage and maintain their files with respect to the diabetic patient. Indeed, it is estimated that less than ten percent (10%) of all physicians use computers in the treatment of their patients for purposes other than billing.

According to the Center for Disease Control (CDC), advances in diabetes research now provide the clinical and therapeutic means to improve outcomes for people with diabetes. The 1993 landmark study, the Diabetes Control and Complications Trial (DCCT), conclusively showed that improved glucose control can retard the onset and progression of diabetes complications affecting the eyes, kidneys, and nerves. A second study in the United Kingdom, entitled United Kingdom Prospective Diabetes Study (UKPDS), released in 1998, confirmed the results of the DCCT and left little doubt about the benefit of lowering blood glucose levels as close to normal as possible. In addition, new medications are available to lower blood glucose and methods for improving glucose levels have greatly improved. The key factor in accomplishing improved results is being able to support the delivery of care that is based on achieving these clear and critical goals.

For providers of diabetes care, these two recently completed studies have now established that there is great personal and economic benefit for diabetic patients to reduce and maintain blood glucose levels as close to normal as possible. For people with Type 2 diabetes, who constitute 90-95% of all diabetic patients, (ADA), aggressive reduction and control of blood glucose levels reduces the risk of blindness and kidney failure by 25%. For patients who also have high blood pressure and aggressively reduce it, major reductions in risk of stroke (44%) and heart failure (56%) can be achieved. (UKPDS Preliminary Results 1998).

With the scientific basis supporting the need for as close to normal blood glucose control now established, the opportunity to improve results begins in an environment that currently falls far short of this goal. The need for great improvement in diabetes care is evidenced by the following assessment from CDC: “Nonetheless, research advances in diabetes are not being communicated effectively and diabetes is not being managed aggressively. The U.S. is far from reaching the objectives set in the U.S. Department of Health and Human Services' Healthy People 2000. Physician practices often do not meet recommended standards of diabetes care. Many patients do not manage their diabetes well. Furthermore, the health care system, which is designed to treat acute and episodic illnesses, is poorly equipped to manage a complex, multi systemic chronic disease like diabetes . . . ”

HEDIS (Health Plan Employer Data and Information Set) serves as the clinical performance measurement and data repository for private and federal health-care buyers. HEDIS is a database of quality measures developed by NCQA and used as a standard evaluation tool for health plans. National quality reporting has established that the patient eye exam, the initial and single standard quality measure for diabetes, is still not completed each year for more than half of all patients. Without tools to plan for the care and to collect and monitor data, diabetes care providers continue to struggle to improve their performance with this single basic measure.

Thus, what is needed is a data processing system and method for managing diabetes care where utilizes known medical standards adopted by the American Diabetes Association, among others, to customize a treatment plan, which can interface with the physician, health care plan and patient, and defines a set of criteria which defines a high risk patient and which continually monitors the patient, setting forth alarms when the patient fails receive a planned examination or service and/or the examination does not fall within an expected range.

### SUMMARY OF THE INVENTION

The present invention is an improvement of the type disclosed in U.S. Pat. No. 6,277,071 to Hennessy et al., also assigned to the assignee of this application. The present invention provides a system that performs real-time monitoring, analysis, and reporting of patient outcomes, illustrating and substantiating the clinical effectiveness of medications, devices, and other chronic disease clinical and educational interventions. This automated process analyzes changes over time in software defined and stored clinical indicators. Outcomes are monitored, analyzed, and reported for single patients or patient populations. Patient data can be aggregated from multiple locations, and standard or custom (both tabular and graphical) reports can be generated. Aggregated data is stripped of patient identifiers to protect confidentiality.

Moreover, the system enables tracking of educational intervention outcomes. By tracking changes in patient clinical measures, the effectiveness of the educational intervention can be measured empirically. The system also automates the development and updating of individualized assessment and educational plans for each patient.

The system produces automated, context-driven patient communications and education. It will produce multi-language correspondence that reminds patients about planned care events and offers results-based feedback about the most recent changes in their glycemic control. Digitally stored education materials are produced and scheduled for patient delivery based on a care plan customized for each patient.

The system integrates comprehensive clinical management with the delivery and assessment of patient education, consistent with the National Standards for Diabetes Self-Management Education and other national standards.

The above discussed and other drawbacks and deficiencies of the prior art are overcome or alleviated by the chronic disease monitor of the present invention.

According to the present invention, there is provided a system for monitoring a chronic disease including a database for storing a plurality of patient data entries. Each of the patient data entries includes personal information of a patient and a set of guidelines concerning the patient's care. A user interface is included for displaying the patient data entries stored in the database and entering the patient entries for storage in the database. A processor retrieves the patient data entries selected by the user interface from the database and stores the patient data entries in accordance to an algorithm. The algorithm comprises a plurality of rules for comparing patient data entries to the guideline to determine whether a test threshold has been exceeded

The processor separates the patient entries designated by the user according to a test threshold stored in the guideline. The test thresholds represent known parameters associated with the chronic disease, such as blood glucose, lipids, liver enzyme, and microalbumin for the disease of diabetes. If the test threshold value derived from the guideline is exceeded, an alert sequence is activated, in which the patient is categorized as a high risk patient, the physician is notified, the patient is notified, the health care provider is notified, and the patient's treatment plan is altered to treat the high risk patient.

In accord with the present invention, an integrated patient monitoring, communication, and education system comprises a database for storing a plurality of patient data entries, each of the patient data entries including personal information of a patient and a guideline concerning the patient's care, the guideline including a default test value associated with monitoring the chronic disease, a user interface for entering patient entries for storage in the database and queries related to patient monitoring, communication and education, and a processor for retrieving the patient data entries from the database in response to the queries from the user interface and storing the patient data entries in the database. The processor provides patient and population outcomes analysis in accordance with an algorithm.

Also in accord with the present invention, a chronic disease outcomes, communication, and education system comprises a database for storing a plurality of patient data entries, each of the patient data entries including personal information of a patient and a guideline concerning the patient's care, the guideline including a default test value associated with monitoring a chronic disease, a user interface for entering patient entries for storage in the database and queries into the database related to patient monitoring, communication and education, and a processor for retrieving the patient data entries from the database in response to the queries from the user interface and storing the patient data entries in the database. The improvement comprises a computer program embodied on a computer-readable medium for operating the processor to provide patient and population outcomes analysis according to an algorithm.

Further in accord with the present invention, a computer program is embodied on a computer-readable medium for operating a computer system including a database for storing a plurality of patient data entries, each of the patient data entries including personal information of a patient and a guideline concerning the patient's care, the guideline including a default test value associated with monitoring a chronic disease, a user interface for entering patient entries for storage in the database and queries into the database related to patient monitoring, communication and education, and a processor for retrieving the patient data entries from the database in response to the queries from the user interface and storing the patient data entries in the database. The computer program includes a subroutine for operating the processor to provide patient and population outcomes analysis according to an algorithm.

Still further in accord with the present invention, a chronic disease outcomes, communication, and education system includes a database for storing a plurality of patient data entries, each of the patient data entries including personal information of a patient and a guideline concerning the patient's care, the guideline including a default test value associated with monitoring a chronic disease, a user interface for entering patient entries for storage in the database and queries into the database related to patient monitoring, communication and education, and a processor for retrieving the patient data entries from the database in response to the queries from the user interface and storing the patient data entries in the database. The improvement comprises a computer data signal embodied in a carrier wave representing sequences of instructions which, when executed by the processor, cause the processor to provide patient and population outcomes analysis according to an algorithm

Still further in accord with the present invention, in a computer processing system, an integrated method for monitoring, communicating with and educating a patient with a chronic disease comprises the steps of storing a plurality of patient data entries, each of the patient data entries including personal information of a patient, storing a treatment guideline defining at least one treatment and a predetermined treatment monitoring threshold for the treatment, measuring a condition of the patient in accordance with the treatment, selectively querying the database for factors related to patient and population outcomes analysis in accordance with an algorithm, and retrieving the patient data entries from the database in response to the queries from the user interface and storing the patient data entries in the database.

### BRIEF DESCRIPTION OF THE DRAWINGS

FIG. 1 is a block diagram of a chronic disease monitor of the type embodying the invention.

FIG. 2 is a block diagram illustrating a patient record.

FIG. 3 is a graphical window displayed to a user entering a patient record.

FIG. 4 is another graphical window displayed to a user entering a patient record for complications.

FIG. 5 is another graphical window displayed to a user entering a patient record for office visits.

FIG. 6 is another graphical window displayed to a user entering a patient record for a patient quality plan.

FIG. 7 is another graphical window displayed to a user entering a patient record for a patient therapy plan.

FIG. 8 is a graphical window displayed to a user entering a provider record.

FIG. 9 is a graphical window displayed to a user entering a health plan record.

FIG. 10 is an algorithm for creating the guideline applied to the patient data record.

FIGS. 11A-11C is an algorithm illustrating the application of the guideline to the patient record.

FIG. 12 is a graphical window displayed to a user entering a risk manager record.

FIG. 13 is another graphical window displayed to a user entering a risk manager record.

FIG. 14 is another graphical window displayed to a user entering a risk manager record.

FIG. 15 is another graphical window displayed to a user entering a risk manager record.

FIG. 16 is a graphical window displayed to a user entering a provider record.

FIG. 17 is a graphical window displayed to a user entering a quality report.

FIG. 18 is a graphical window displayed to a user entering a high-risk patient report.

FIG. 19 is a graphical window displayed to a user entering a quality report by provider.

FIG. 20 is a graphical window displayed to a user illustrating warning symptoms and signs for diabetic foot problems.

FIG. 21 is a graphical window displayed to a user entering a patient history record.

FIG. 22 is a block diagram representing the logic sequence for generating a high-risk rule.

FIG. 23 is a continuation of the sequence of FIG. 22.

FIG. 24 is a block diagram representing the logic sequence for a generation liver enzyme testing high-risk rule.

FIG. 25 is a block diagram representing the logic sequence for a patient outcome analysis.

FIG. 26 is a block diagram representing the logic sequence for patient communications.

FIG. 27 is a block diagram representing the logic sequence for patient education.

FIG. 28 is a graphical window displayed to a user for entering a patient medication record.

FIG. 29 is a graphical window displayed to a user for entering clinical measure criteria for a patient.

FIG. 30 is a graphical window displayed to a user showing the effects of the clinical measure criteria of FIG. 26.

FIG. 31 is a graphical window displayed to a user showing a medication outcome for a selected medication.

FIG. 32 is a graphical window displayed to a user showing another medication outcome for another medication.

FIG. 33 is a graphical window displayed to a user showing an outcome for patient weight versus educational intervention.

FIG. 34 is a graphical window displayed to a user showing patient information.

FIG. 35 is a graphical window displayed to a user showing an education content summary for patient medical history.

FIG. 36 is a graphical window displayed to a user showing an education content summary for patient diabetes history.

FIG. 37 is a graphical window displayed to a user showing an education content summary for patient social history.

FIG. 38 is a graphical window displayed to a user showing an education content summary for patient education intervention.

FIG. 39 is a graphical window displayed to a user for entering an intervention.

FIG. 40 is a graphical window displayed to a user showing an education content summary for patient evaluation progress.

FIG. 41 is a graphical window displayed to a user showing further details of the education content summary for the patient evaluation progress of FIG. 38.

FIG. 42 is a graphical window displayed to a user for entering a diabetes registry.

FIG. 43 is a graphical window displayed to a user for entering information in a diabetes registry.

FIG. 44 is a graphical window displayed to a user for entering information regarding an educational class.

FIG. 45 is a graphical window displayed to a user for showing information regarding class schedules.

FIG. 46 is a graphical window displayed to a user for showing information regarding class instructors.

FIG. 47 is a graphical window displayed to a user for entering more detailed information regarding the instructors of FIG. 44.

FIG. 48 is a graphical window displayed to a user for showing information regarding class attendance.

FIG. 49 is a graphical window displayed to a user for creating patient communications.

FIG. 50 is a graphical window displayed to a user for showing patients selected by certain criteria.

### DETAILED DESCRIPTION OF THE PREFERRED EMBODIMENTS

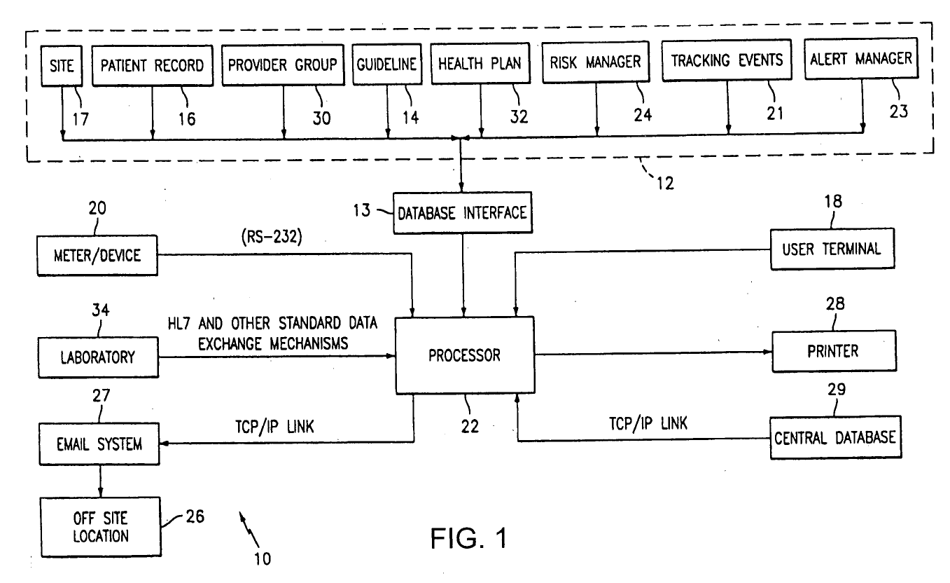

Referring to FIG. 1, a chronic disease monitor in accordance with a preferred embodiment is generally shown at 10. Chronic disease monitor 10 includes a central database 12 that electronically stores chronic disease information and enables a system user to access the stored information to monitor a chronic disease. Central database 12 includes computer memory in the form of RAM and ROM memory and is located in the computer hardware or deposited on a readable storage media. Guideline 14 comprises an algorithm representing known parameters of a chronic disease, including risk factors and complications associated with that disease, may be tailored by the medical provider to implement a facility wide treatment plan to a given patient population as well as on an individual patient basis. Patient record 16 information, such as demographic information 100 and insurance information 102, is inputted by user at user terminal 18, such as a computer terminal, a personal computer interfaced within a local area network, and the like. Site information 17 comprises data associated with the location of the installation (e.g. location, licensee, etc.). Patient information 16 is updated in a variety of ways. For example, a user may enter progress notes and/or test results at user terminal 18. Meter device 20, such as a blood glucose monitor, may provide test results in electronic data form. Processor 22 comprises a central processing unit, such as a microprocessor, which stores and accesses the information in central database 12 (such as a patient record 16). Database interface 13 comprises a plurality of operating systems and programs allowing monitor 10 to store and retrieve data stored in database 12. Patient record 16 is applied to an algorithm within guideline 14. If a test result exceeds an expected threshold, an alert is generated and a notation is stored in risk manager 24. The alert may be communicated to an off site location 26, e.g. via e-mail 27, such as to an employer, health maintenance organization and the like, and/or a letter may be printed to the patient via printer 28. Additionally, if a patient fails to attend a scheduled service, an alert is similarly generated. Processor 22 may optionally be linked to a central database 29 (offsite) via a TCP/IP link as is known in the art.

Provider information 30 (e.g., a physician) and health plan information 32 are also stored in central database 12, to enable communication with medical providers and third parties. While the chronic disease monitor of the present invention may be used for other chronic diseases, chronic disease monitor 10 is particularly relevant with respect to diabetes and therefore, hereinafter, the chronic disease monitor will be described with respect to the monitoring and control of diabetes.

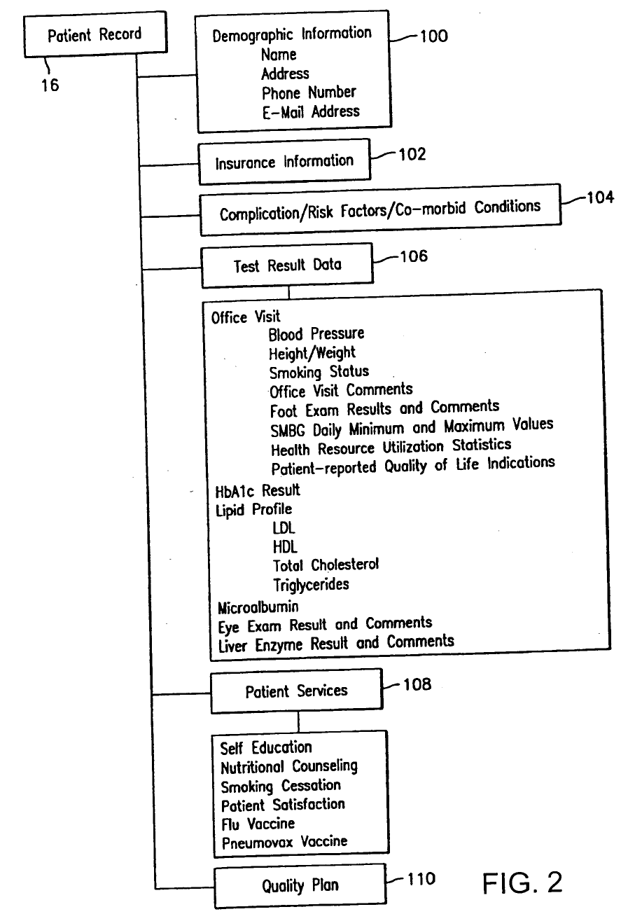

Referring now to FIG. 2, patient record information 16 is generally shown in block diagram form and is described as follows. Monitor 10 incorporates a window format and is programmed in Microsoft Visual Basic® to operate in a Windows® environment. It will be appreciated by those of ordinary skill in the art that other programming formats and/or languages may be employed. Patient record 16 is entered by a user at user terminal 18 and includes the patient's demographic information 100 e.g., salutation, name, gender, year of diagnosis, diabetes type (type 1, type 2, gestational), address, contact information (e-mail, work and home phone), initiation of care date, health plan, health plan id, provider, employer and language. Insurance information 102 is also recorded in patient record 16. An identifying number for the patient is stored in the database. Additionally, complications, risk factors/co-morbid conditions 104 such as retinopathy, neuropathy, nephropathy, PVD, CAD, and cerebral vascular disease are recorded.

Patient record 16 also include test data 106. Test data 106 comprises the office visit date, practitioner, office visit comments, such as progress notes and patient concerns, are recorded. Clinical information, i.e. weight, height, blood pressure, smoking status, blood glucose recordations (SMBG), lipids profile, liver enzyme, foot exams, neuropathy, skin condition, eye exam, are stored. It will be appreciated to those skilled in the art that the blood glucose information maybe entered manually or electronically transferred from a blood glucose-metering device 20, such as a Life Scan OneTouch. Data may also be transferred directly from a laboratory, such as via an RS-232 port or TCP/IP (FIG. 1) in HL7 (or other standard data format). Quality of life indicators, such as number of emergency room visits, days of hospitalization, days lost from work, and activities, provide important outcome information. By storing this information in patient record 16, reports may be generated comparing changes in these factors over a given period of time and/or for a selected treatment therapy. Combinations may be applied. Further, a patient's own self-assessment is recorded as diabetes is such that success in treatment is heavily dependant on the patient's active participation.

Patient record 16 also includes a quality plan 110. Monitor 10 generates quality plan 110 from a selected guideline 14 and allows the user to customize the quality plan by selecting frequencies, thresholds and goals for a series of tests, which are required to be performed on the patient, setting alert values if thresholds are exceeded or if tests are not undertaken. For example, tests for HbAlc, lipids (to measure cholesterol), blood protein (microalbumin), eye and foot examinations are recommended by the American Diabetes Association. As described in greater detail below, the frequencies for these examinations are defaulted to the recommended ADA values (but may be over-written by the user). Additional tests may be programmed, such as a stress test for cardiovascular disease. The frequency of office visits may be stored. Monitor 10 notifies providers, health care plans, and patients via letters, e-mail, etc. Letters may be stored in the form of reminders, and/or report letters, indicating test results, a missed appointment, an alert and the like. Patient services 108 including self-education, nutrition counseling, smoking cessation; patient satisfaction, flu vaccine, and pneumonia vaccine are also stored in patient record 16. Patient record 16 also includes a patient's medications, therapies, and treatments (such as medication, dosage, frequency start date, a nutrition plan and exercise plan).

It will be appreciated by those of ordinary skill in the art that the window environment allows the user to access this information from window to window and that additional information may be optionally stored “behind” the window in layered fashion.

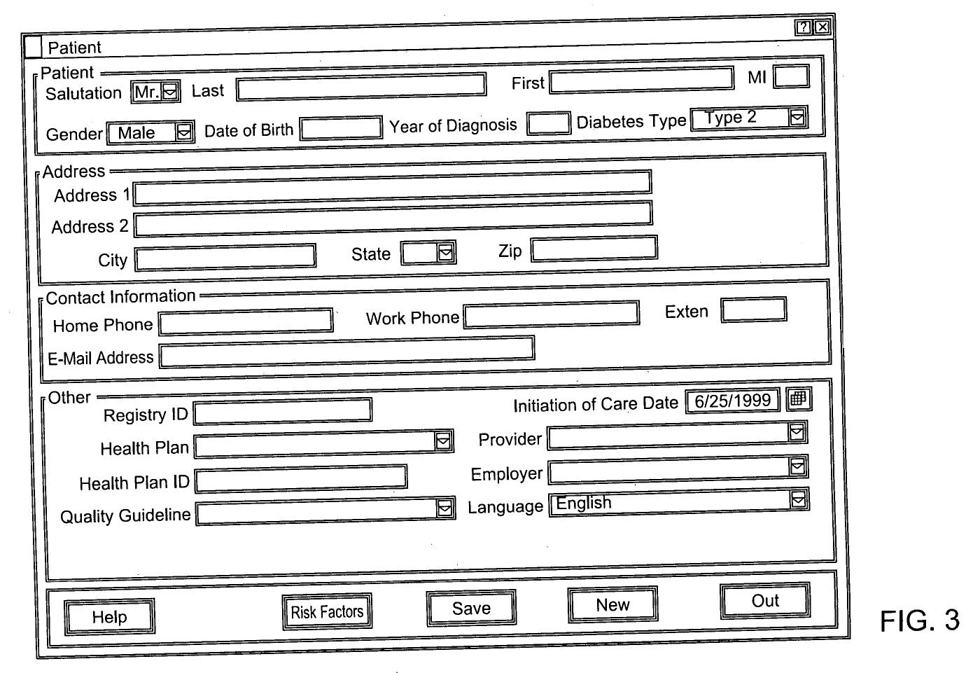

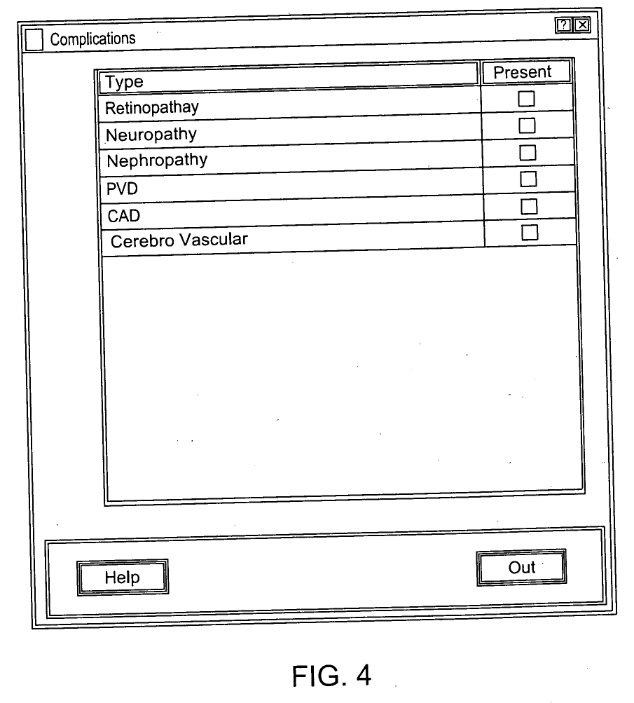

As shown in FIGS. 3 through 7, patient data 16 is presented to a user in a window format, though other known program formats may be used. FIG. 3 illustrates the patient setup, where the user may input the patient's demographic information 100, such as salutation, name, gender, date of birth, year of diagnosis, diabetes type, address, contact information, registry ID, health plan, quality guideline, provider, employer and language. FIG. 4 illustrates a second portion of the patient setup where the user may select complications, risk factors/co-morbid conditions 104 which the patient suffers, such as retinopathy, neuropathy, nephropathy, PVD, CAD and cerbro vascular diseases.

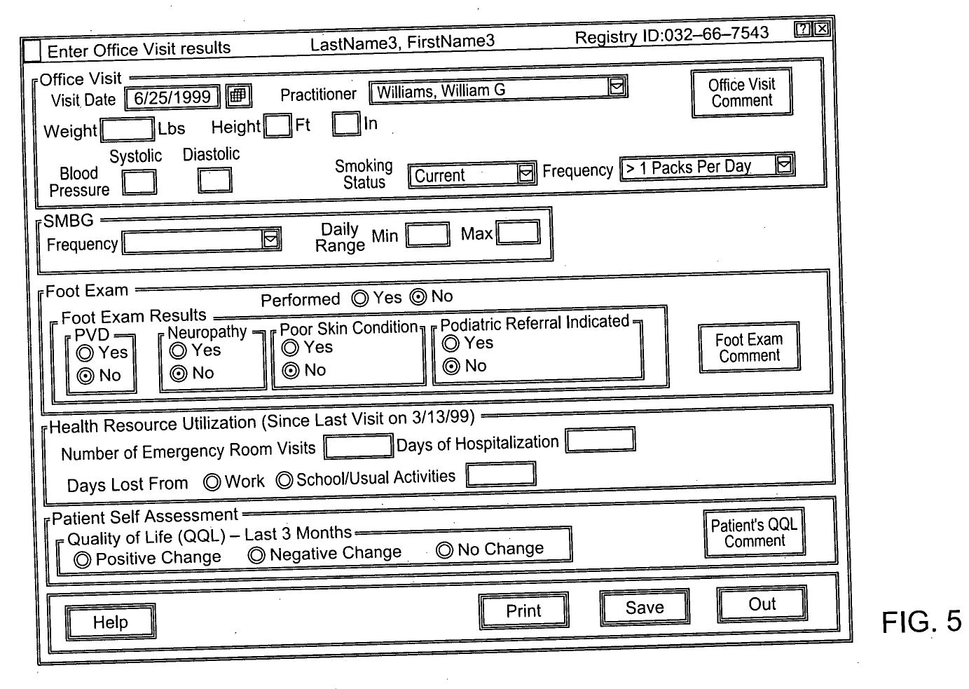

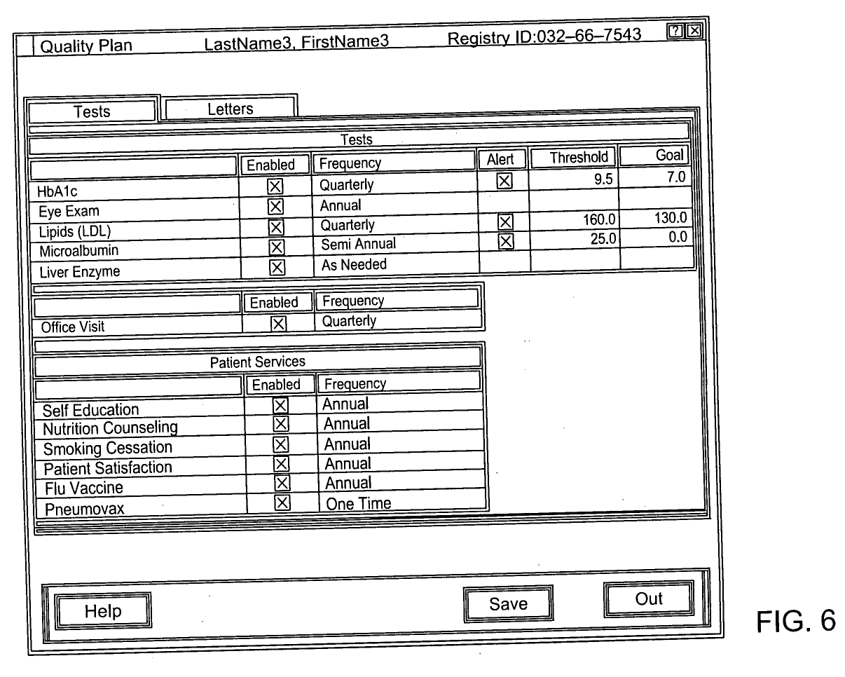

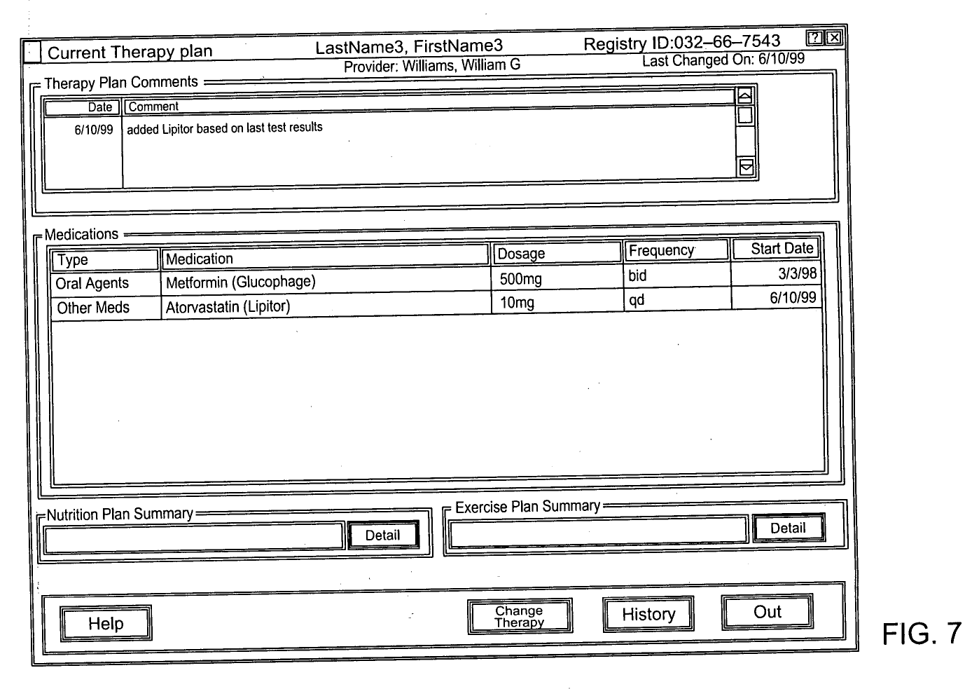

FIG. 5 illustrates a window, which is prompted when office visit data is entered into patient record 16. The user may enter the office visit date, practitioner, weight, height, blood pressure, smoking status, blood glucose (SMBG) and daily range, foot exam (PVD, neuropathy, poor skin condition, podiatric referral), quality of life indicators (number of emergency room visits, days of hospitalization, days lost from work) and the patient self assessment. FIG. 6 illustrates a window, which is prompted for the creation of a patient quality plan 110. The tests to be preformed on the patient are selected for enablement, frequency, alert (where a value is exceeded), threshold, and goal. As described in greater detail below, the values for the threshold default to the guideline value located in guideline 14 generated for the patient population in risk manager 24. The user may enter a different value for a given threshold and override the guideline default. The user is prohibited from entering a threshold value which would be impossible (outside of permissible test ranges, for example) and which is greater than the patient population threshold. FIG. 7 illustrates a current therapy plan data record in patient record 16 as presented to the user in a window format. The user may input comments. The information is classified by medication type, medication, dosage, and frequency and start date. The nutrition plan summary and/or exercise plan summary may also be entered.

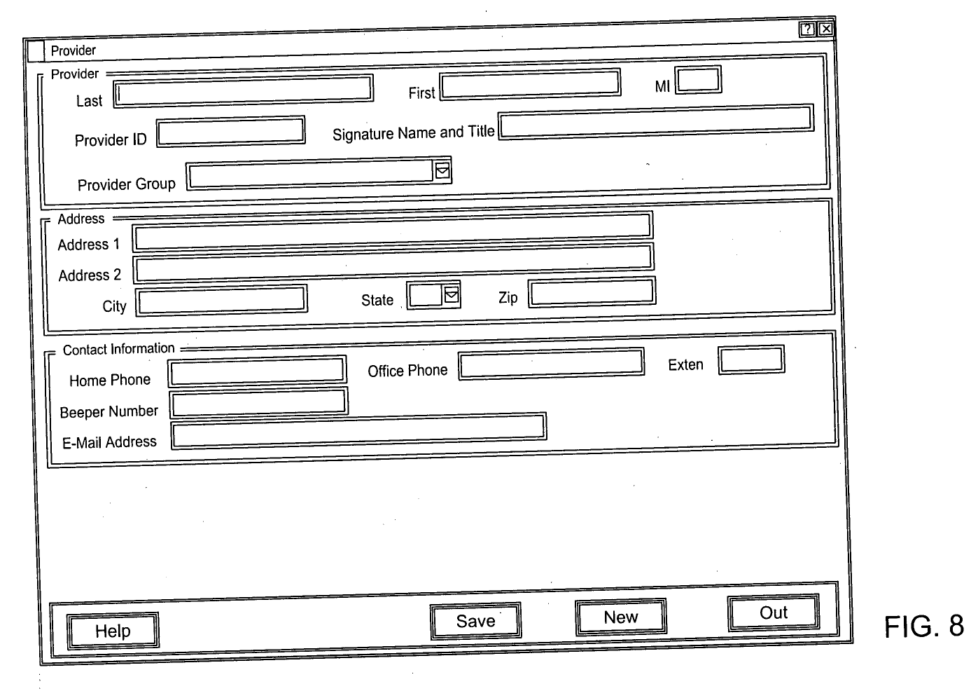

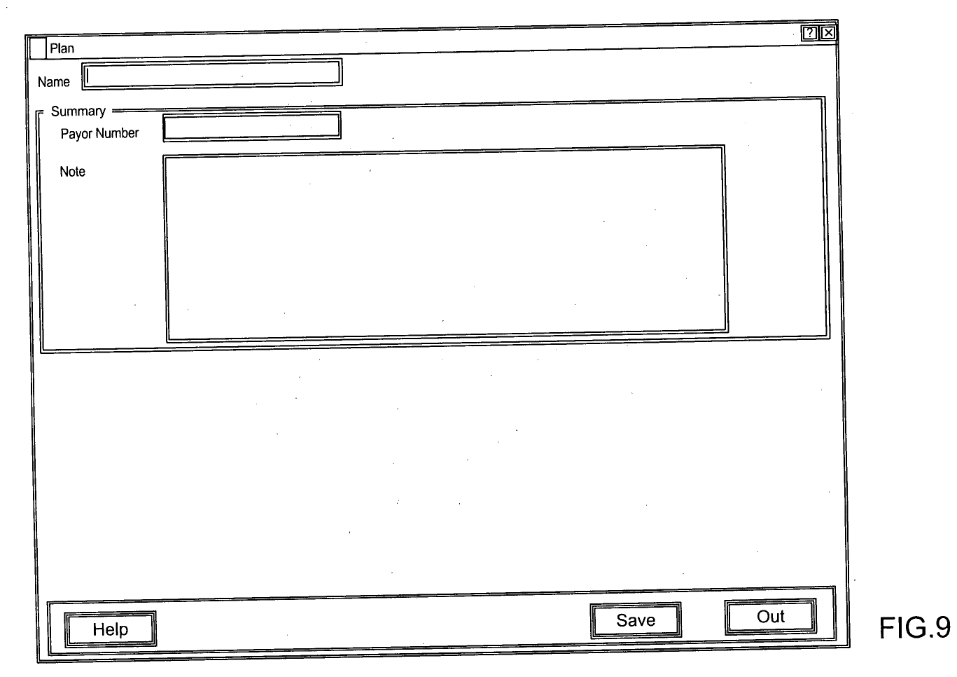

Referring now to FIG. 8, provider information, such as for a physician, including the name, address, identification number, contact information, beeper number and the like stored in database 12 as provider record 30 is generally illustrated. The user is prompted with a window, which allows the user to enter the information. FIG. 9 illustrates a window for receiving health plan records 32. The user may input comments concerning specific policies, which are recorded in health, plan record 32.

Referring again to FIG. 1, guideline 14 comprises an algorithm, which represents the diabetic treatment model recommended by the ADA. Guideline 14 represents the recommended tests (and frequencies), alert thresholds, and goals for the care of the diabetic patient. A user may use an ADA default, may program a different set of thresholds for a patient population, and may adjust the parameters for each patient to establish a quality plan. The ADA publishes standards entitled HEDIS (Health Plan Employer Data and Information Set) 3.0, HEDIS 99, and the ADA Provider Recognition Program. These standards contain recommended (and accepted) treatment schedules for the diabetic patient. For example, HEDIS 99 requires quarterly HbAlc testing, annual eye exams, annual lipid profile, and annual microalbumin exams. Typically, the alert threshold for the HbAlc test is 9.5%, with a goal of at least 7.0%. Similarly, the annual lipid profile typically has a goal of 130 Mg/dl and an alert threshold of 160 Mg/dl. Microalbumin exams have a goal of 9, with an alert threshold of 25 Mg/L. Thus, these parameters are incorporated into a rule structure for the monitoring of the diabetic patient. As described in greater detail below, the user may select a rule for the treatment of the patient population. For example, the data may sorted for all patients having hypertension and having a blood glucose test level exceeding 9% HbAlc. For each patient data entry, a comparison is made between a guideline value (measure value) in guideline 14 and the test data from patient record 16 to determine if the rule is satisfied and/or whether a threshold level has been exceeded.

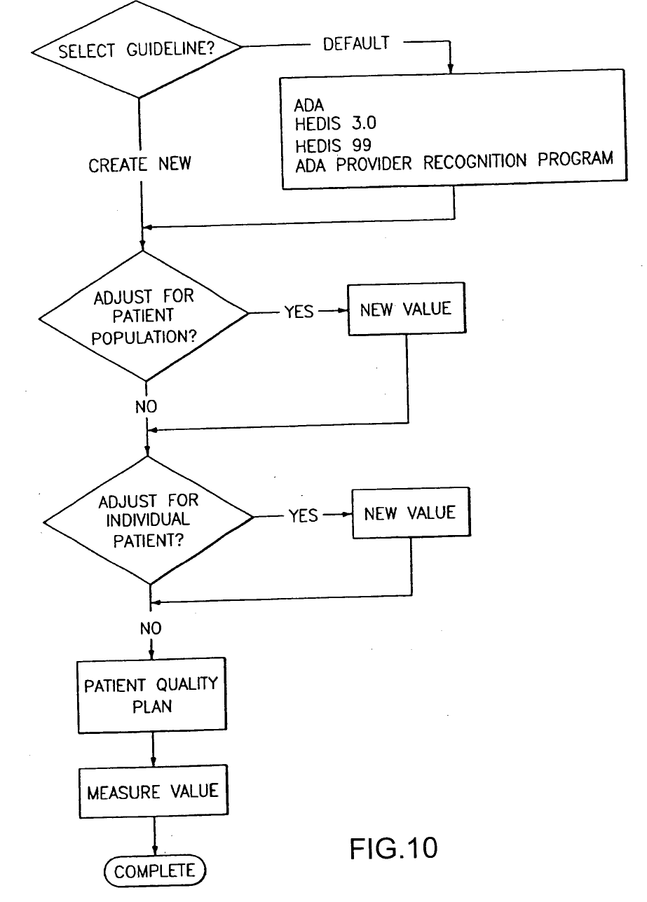

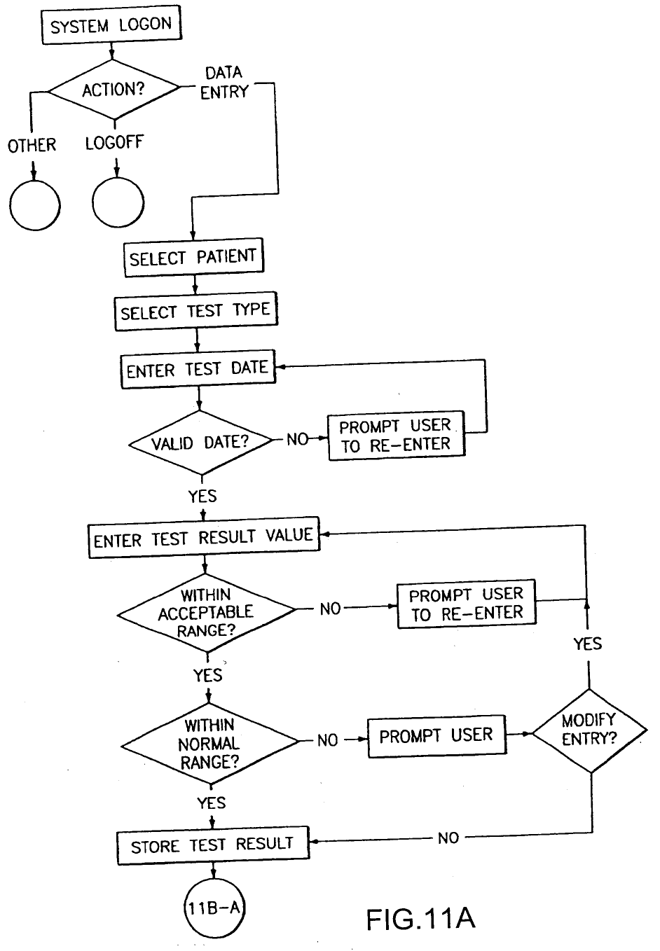

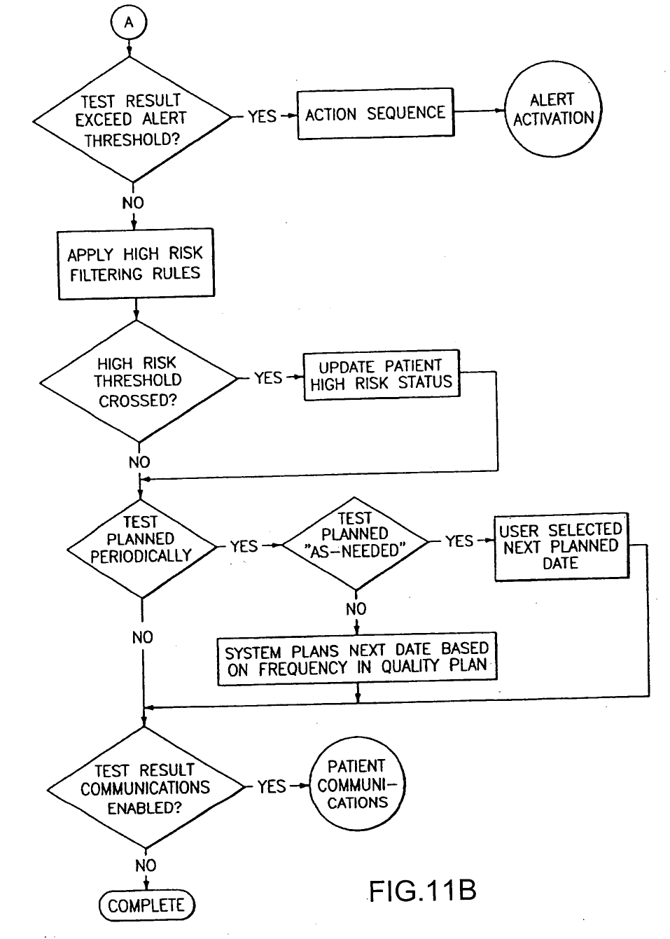

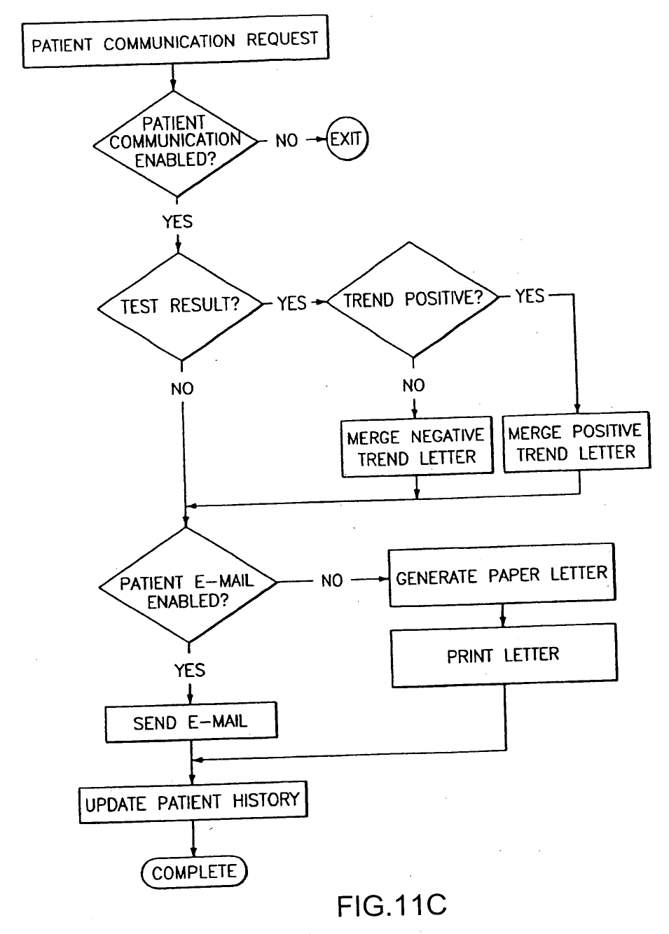

Turning now to FIGS. 10, 11A, 11B, and 11C, the logic sequence of guideline 14 and risk manager 24 will be explained in greater detail. The ADA has published recommended guidelines for the treatment of diabetes. These guidelines are based on years of study of the disease and comprise the recommended treatment for individuals suffering from the disease. For example, the HEDIS 99 guideline sets as an alarm threshold for microalbumins greater than 25 Mg/L, which indicates a patient who requires immediate medical assistance. This value is stored in guideline record 14 as a default value. The user may select a default guideline, such as the ADA HEDIS 3.0, HEDIS 99, and/or ADA Provider Recognition Program. Alternatively, the user may create a guideline for any and/or all of the measures (HbAlc, eye exam, lipid (LDL), microalbumin, liver enzyme, self-education, nutrition counseling, smoking cessation, patient satisfaction, flu vaccine, and pneumonia vaccine. The user may also modify the ADA rule and enter a new threshold limit in place of the ADA guideline default in guideline 14 for a given patient population, e.g. for all patients covered under a certain plan, associated with a certain physician, and the like. Additional rules, representing threshold values, may be selected. For example, patients with HbAlc value of greater than 9.5%, or patients who have not been tested, who also have an associated risk factor for hypertension, may be selected by the user to create a rule within guideline 14 to be applied to the database 12. A HbAlc reading of 9.5% or a patient having the risk factor of hypertension would mean that the patient is at high risk and needs immediate medical treatment. Incorporated in the guideline 14 are known parameters for the disease of diabetes so that a user may not input a value which would be outside of possible testing values. If the ADA guideline value is not adjusted, then it will be the default value for the individual patient for the generation of a patient quality plan. Alternatively, the patient population default value may be adjusted for an individual patient for incorporation into the patient's quality plan. The value assigned in the patient quality plan is the measure value against which test results and clinical events are compared.

A test result may be communicated from Laboratory 34 via an RS-232 port directly to the processor 22, may be blood glucose data generated from a hand held blood glucose meter device 20, and/or may be manually inputted by a user at user terminal 18 and recorded in record 16. A clinical finding or notation, such as a missed service, a new complication, a measurement and the like may also be entered and stored to be applied to the guideline 14. The value is compared against the measure value derived from the patient's quality plan 110. If the test result/clinical event exceeds the expected measure value (or a scheduled service or event is missed or omitted), a series of program functions are performed. The functions resulting from the threshold value being exceeded and/or from the application of a rule may be generally described as an action sequence. Whether the action sequence is activated or no action is taken, the patient's record 16 is updated to reflect the test result/clinical event. If the alert function has been selected in the patient record 16, an alert for the patient to alert the system manager and/or medical provider/physician of the event is registered. Also, the patient's name is added to the risk manager 24, a letter is generated to send to the patient (and/or another physician or caretaker), the information is communicated off site 26, such as to a health maintenance organization, provider, and the like. Also, the patient's quality plan 110 in patient record 16 may be updated to reflect the necessity for additional tests and/or a different frequency or parameter for the tests as a result of the exceeded threshold. The clinical schedule is also updated to reflect the patient's need for additional services. Alternatively, a report letter, with encouragement concerning the test result, and/or explaining the test result, may be sent to the patient.

Referring now to FIGS. 11A through 11C, the user logs onto the system from user terminal 18 and selects an action. The user may access the data records stored on central data base 12 and/or may chose to enter data (a patient record 16 is then selected). The test type is selected. The test date is then entered. If the date is invalid, i.e. a date in the future or an impossible date, e.g. 1867, Monitor 10 will prompt the user to reenter. The user then enters the test value. If the test value is outside an acceptable range (known physical parameters) the user is prompted to again reenter. Similarly, if the test value exceeds normal values, but possible values, the user is prompted to confirm the value. If the data is entered via a meter/device 20 or other electronic device, an alert report is generated (if the data exceeds known parameters). After the data is stored it is compared to the guideline value (measure value). As set forth above, the guideline value may be a threshold for a test result and/or may comprise a rule combining a test threshold and a complication. If the threshold is exceeded and/or if the rule is satisfied, the action sequence is undertaken (i.e. alert, letter, offsite, update quality plan, update clinical schedule, add to risk manager, etc.). The data is then measured against the high-risk monitor. If the high-risk threshold is crossed, the high-risk status is updated. Next, the planned events data within the patient record 16 is updated based on the criteria for the quality plan. For example, if the test is scheduled “as needed,” the user is prompted to select a date. If there is another selected frequency for the test, such as quarterly, Monitor 10 will automatically schedule the test. If the test result communications is enable, the patient then receives notification of the test result and/or new test date. If it is a test result communication, the trend is determined (positive/negative). Next, the patient e-mail enablement is determined. If yes, the communication is e-mailed. If no, a letter is generated. Patient record 16 is then updated.

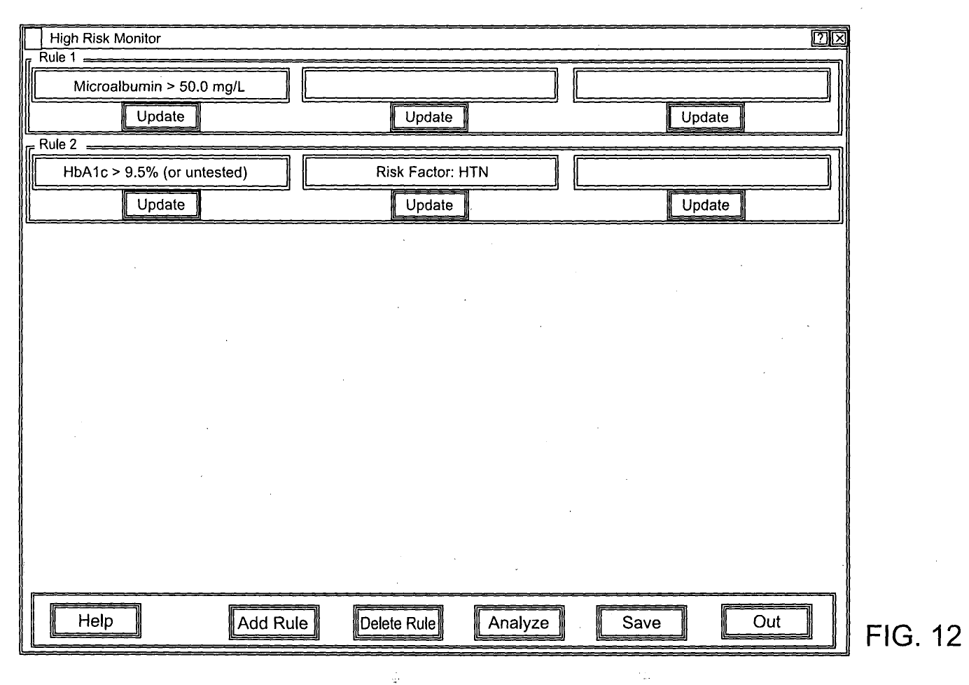

Thus, for example, a user selects the high-risk monitor in the menu driven format at user terminal 18 and adopts a rule for HbAlc ≧9.5% (or untested) for patients with the risk factor of hypertension. As shown in FIG. 12, which illustrates the data entry prompt for the user when accessing the risk manager 24, the user may create a series of rules to be applied to a patient population by entering a threshold, risk factor and the like. For example, as illustrated in FIG. 12, if Rule 1 set the threshold for microalbumin levels >50.0 mg/L and Rule 2 set HbAlc >9.5% (or untested) for patients with hypertension, if either condition is met, the patient's name will be added to the high risk patient list (and the action sequence will be applied). Thus, if the microalbumin level exceeds 50 mg/L (Rule 1) or if the HbAlc test value exceeds 9.5% and the patient has the complication of hypertension (Rule 2), or if the patient has not been tested (Rule 2), the patient's name is added to risk manager 24. The action sequence is also initialized (i.e., alert, quality plan is updated to reflect the need for additional services, such as greater frequency in testing blood glucose, information is sent off site 26 to a pay provider, employer, health maintenance organization and the like, a letter is generated to the patient, and the appropriate physicians receive an alert concerning the test result/clinical event). If a test result/clinical event was less than the measure value of less than 50.0 mg/L microalbumin (Rule 1) and 9.5% for HbAlc (Rule 1), patient record 16 is updated and the action sequence is not executed.

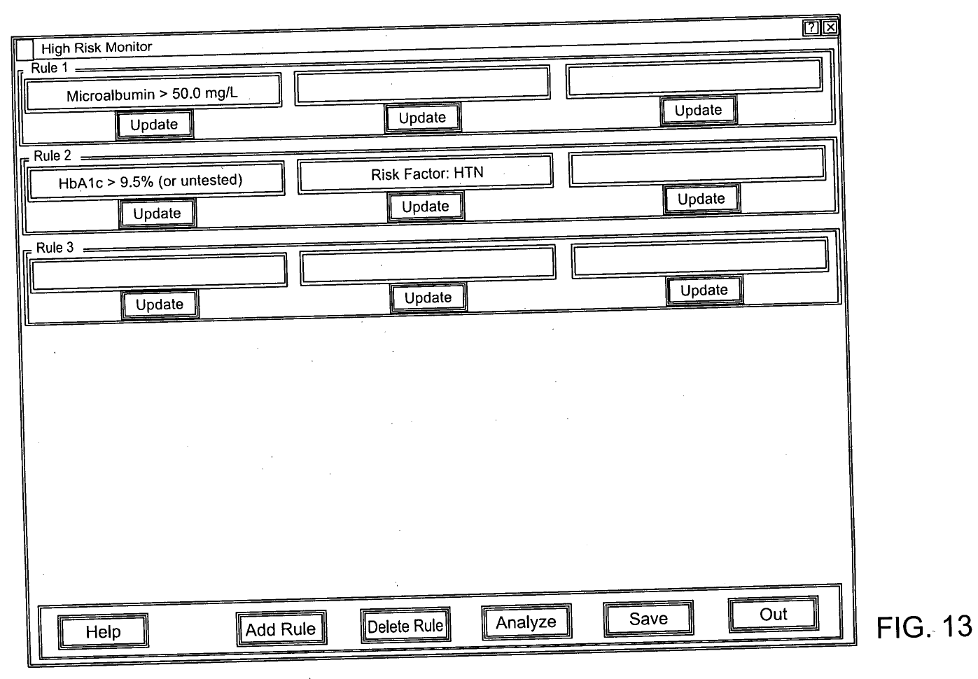

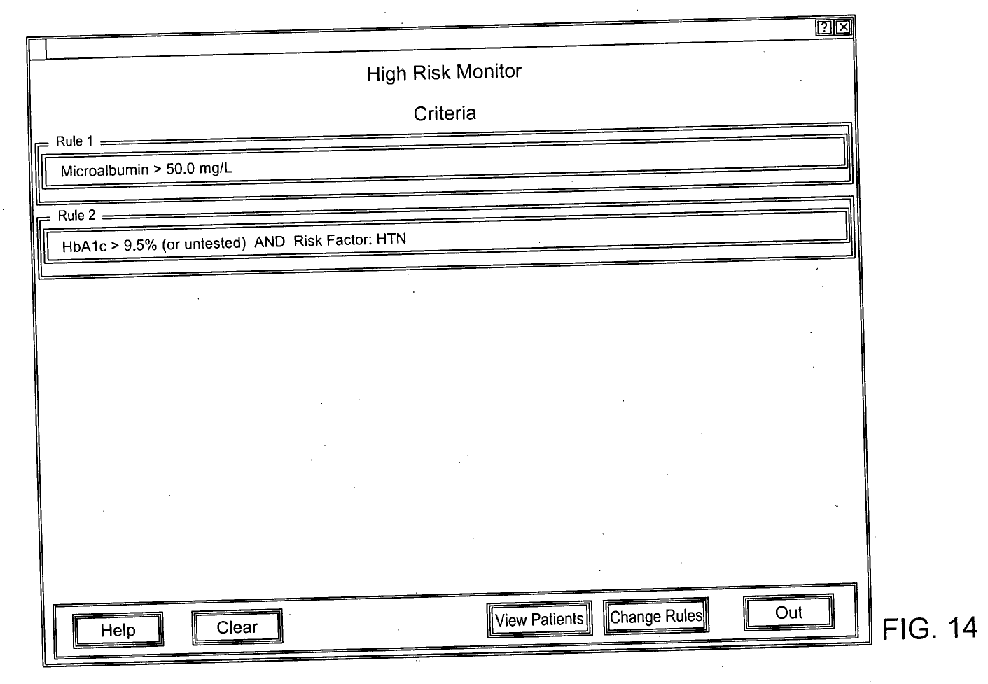

Referring to FIGS. 13 and 14, risk manager 24 data is inputted by a user at user terminal 18. A window may be employed to prompt the user with respect to the data to be entered. The user may add a rule, deleted a rule, analyze the patient records 16 by rule, and change a rule.

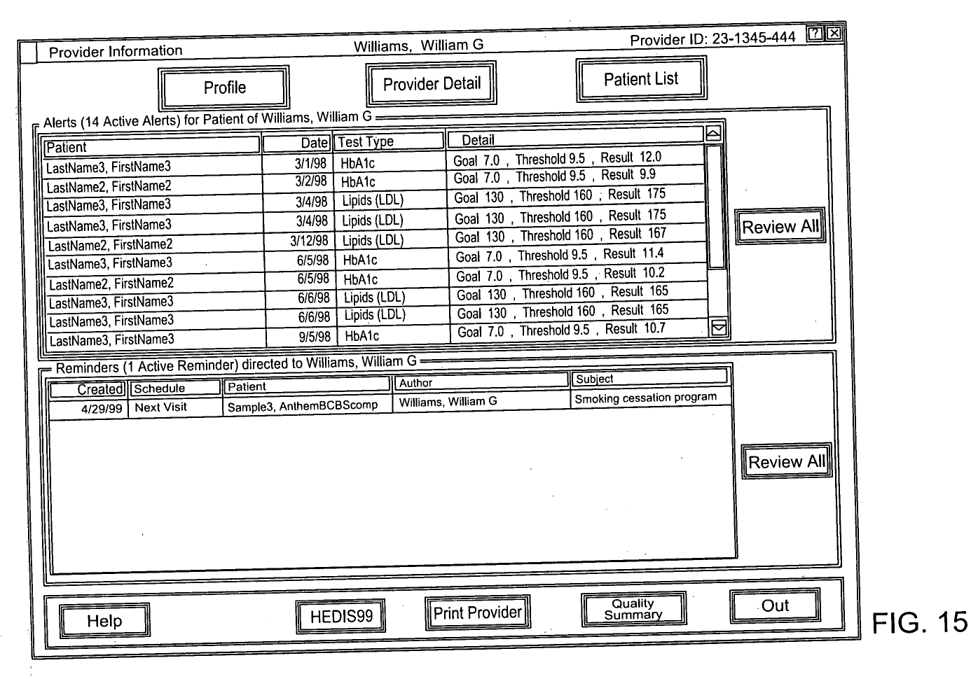

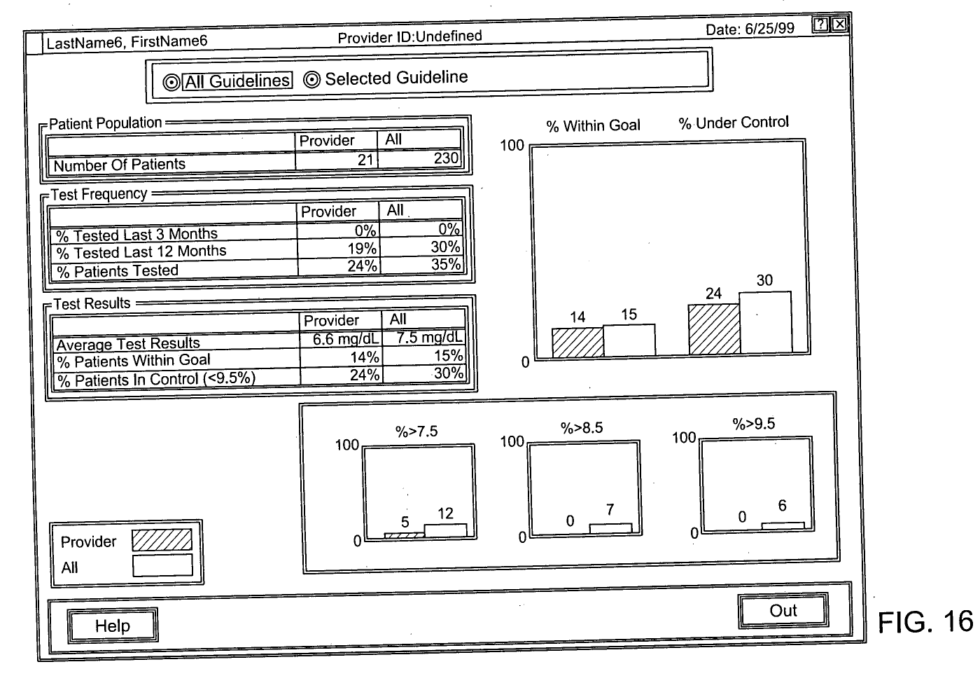

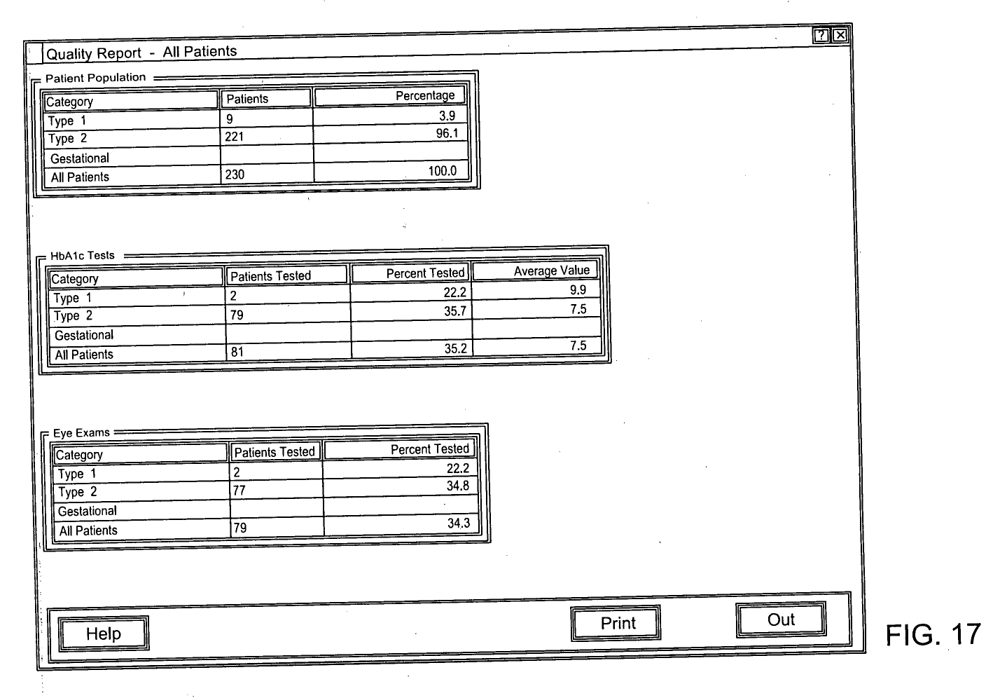

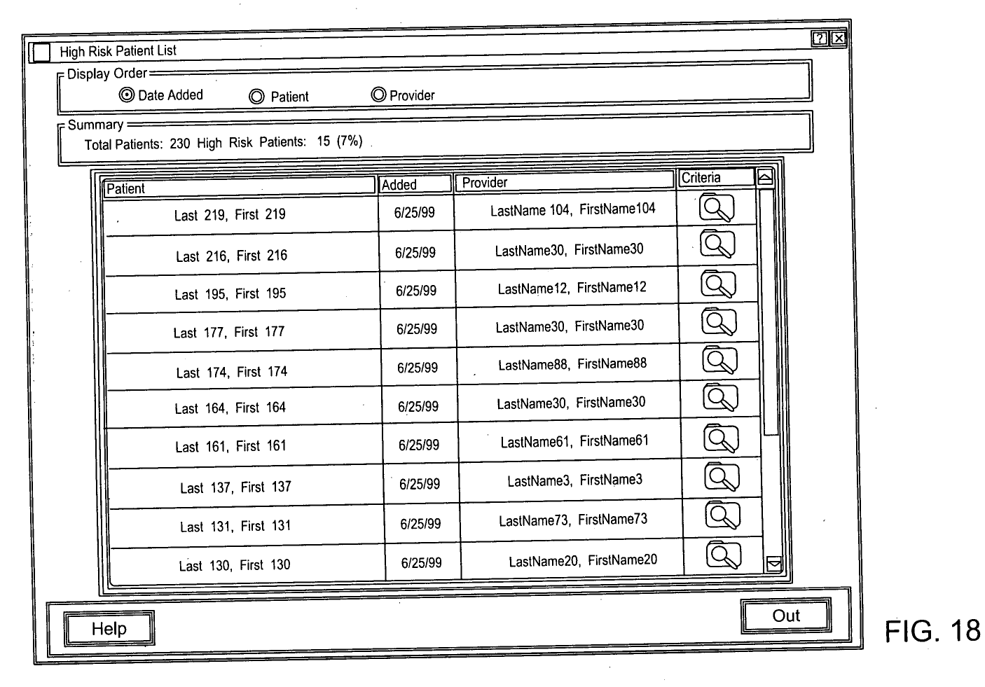

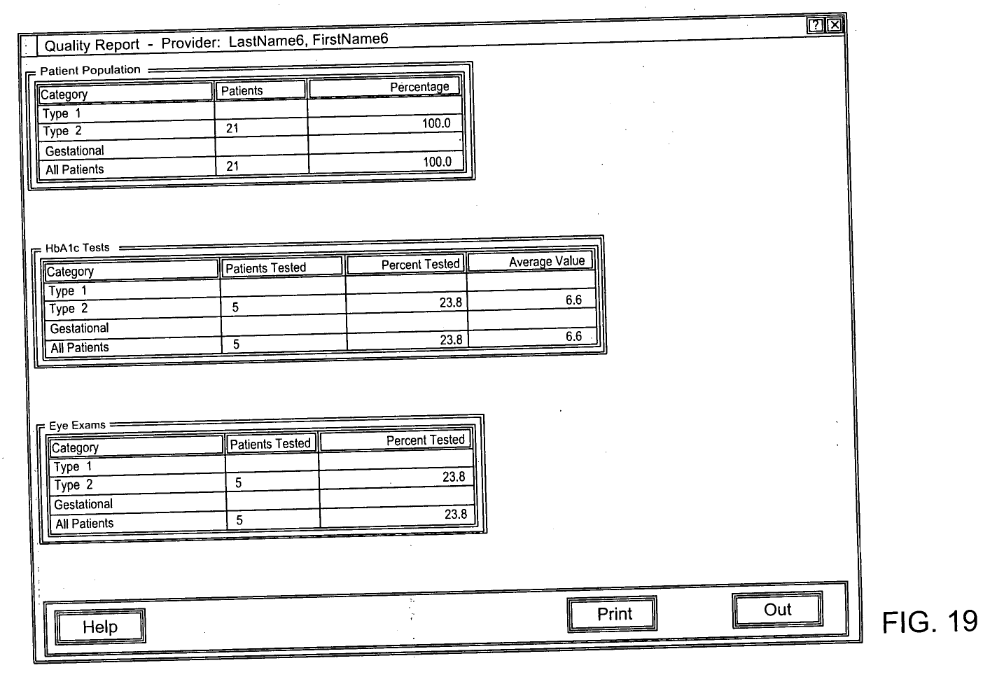

Referring to FIG. 15, an example of the type of report, which may be generated as a result of the chronic disease monitor 10, is illustrated. It will be appreciated to those of ordinary skill in the art, that by applying the guideline and logic sequence described herein, that various reports may be generated to assist the physician, patient, and/or pay provider to monitoring the chronic disease. The user, via user terminal 18, selects the menu setting forth the provider record 30, which associates information with respect to a medical provider such as a physician. For the physician, the alerts are categorized by patient, date, test type, detail (goal, threshold, result). Reminders are also listed for the respective physician, indicating the date created, schedule, patient name, author, and the subject. As illustrated in FIG. 16, the patient population may be viewed globally, settling forth the number of patients seen by the provider, the test frequency, test results, with graphical illustrations. Quality reports for patients, setting forth the patient population, including the category of diabetes, the number of patients and percentage of the patient population, the tests undertaken on the patient population, and the average result of those tests, are illustrated in FIG. 17. As shown in FIG. 18, a high-risk patient list can be generated for distribution to providers, payers, etc. The background criteria are stored behind each high-risk patient in window format. As illustrated in FIG. 19, a quality report by provider, setting forth the category, patients, percentage of patients by diabetes type, test (eye exam, foot exam, lipids, etc.) may be accessed.

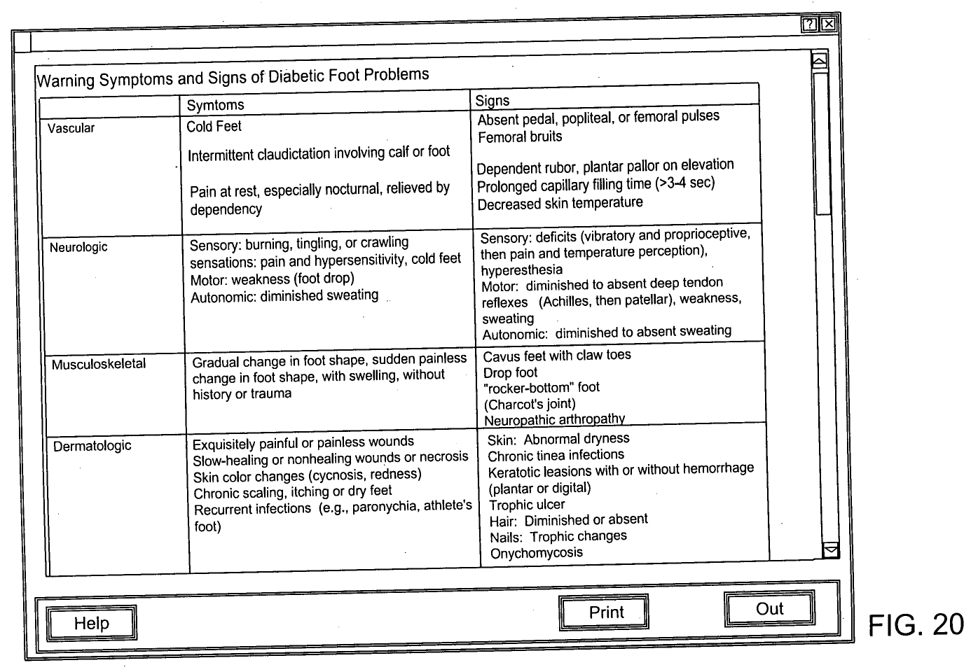

As shown in FIG. 20, additional information, such as warning signs concerning symptoms and signs of foot disease may be stored (and optionally communicated to the patient).

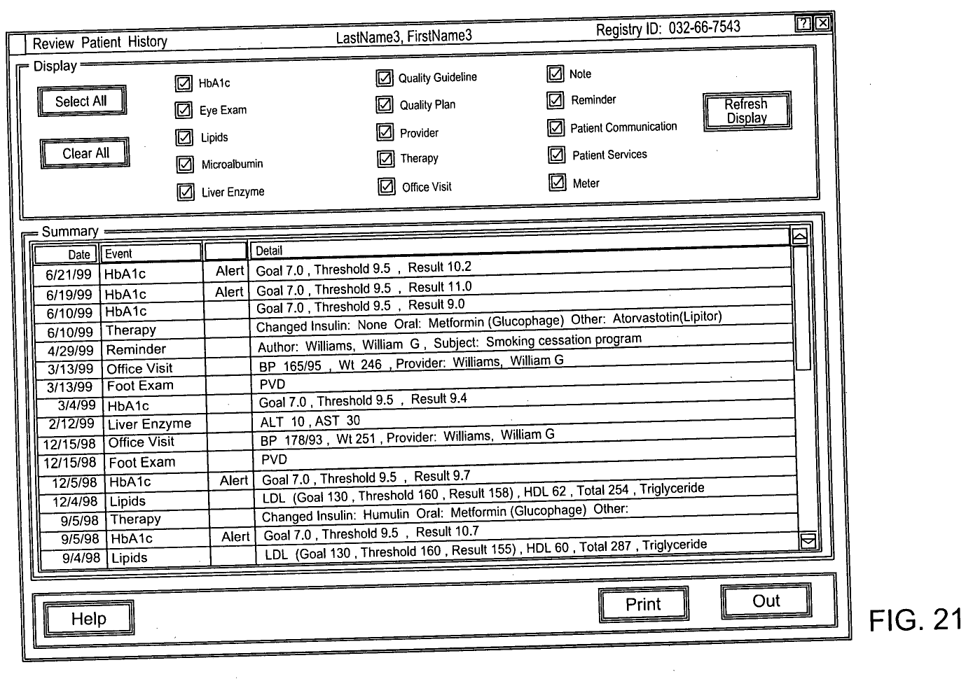

As illustrated in FIG. 21, a report representing the patient's history using chronic disease manager 10 is shown. The information is categorized by date, event, and detail. If an alarm has been generated, it is also illustrated. For example, if the patient goal for HbAlc, was 7%, and the test result value was 20%, applying the logic sequence as herein described using the guideline algorithm results in the action sequence causing the generation of the alert and the placement of the patient's name in the risk manager. The user may elect to see all entries. Alternatively, the user may select test results for a given patient by category, i.e. HbAlc, eye exam, lipids, microalbumin, may view quality guideline, quality plan, therapies, office visits, notes, reminders, patient communications, and meter selection by entering a check, such as with a mouse.

The user may also elect to change the rule and/or threshold in the risk manager. If so, the user will receive a warning, advising the user that the high-risk patient list will be changed and the previous high-risk patient list will be deleted. The user will receive a prompt, asking if the user wishes to continue. The user may elect not to continue and may then select another option in the risk manager or exit to select another function. If the user changes the threshold value (and/or rule) such as for lipids, HbAlc, etc., the new threshold, rule, and/or Boulian combination thereof, will then be compared to the patient data. If the value is exceeded for the patient (or if the rule is satisfied), the action sequence is undertaken, and the alert, letter, offsite, clinical schedule, and risk manager are updated.

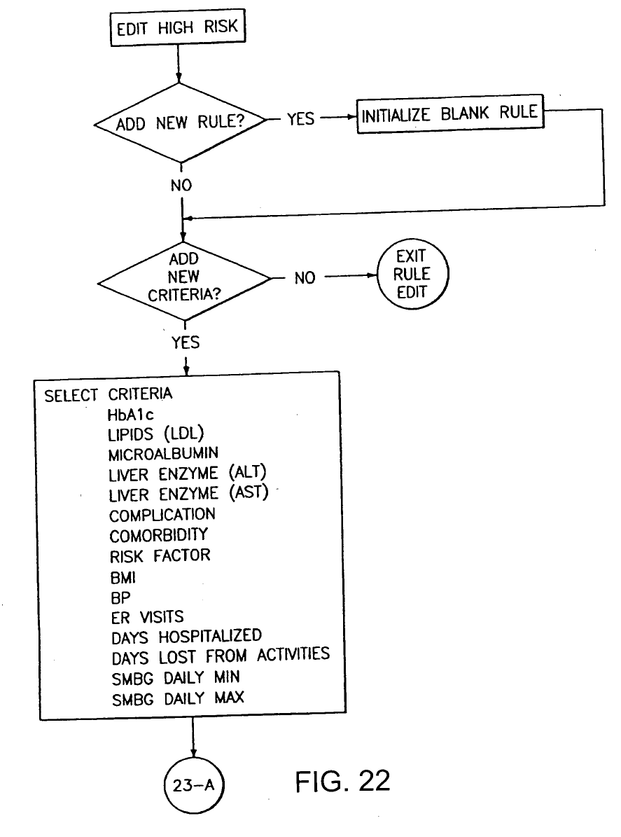

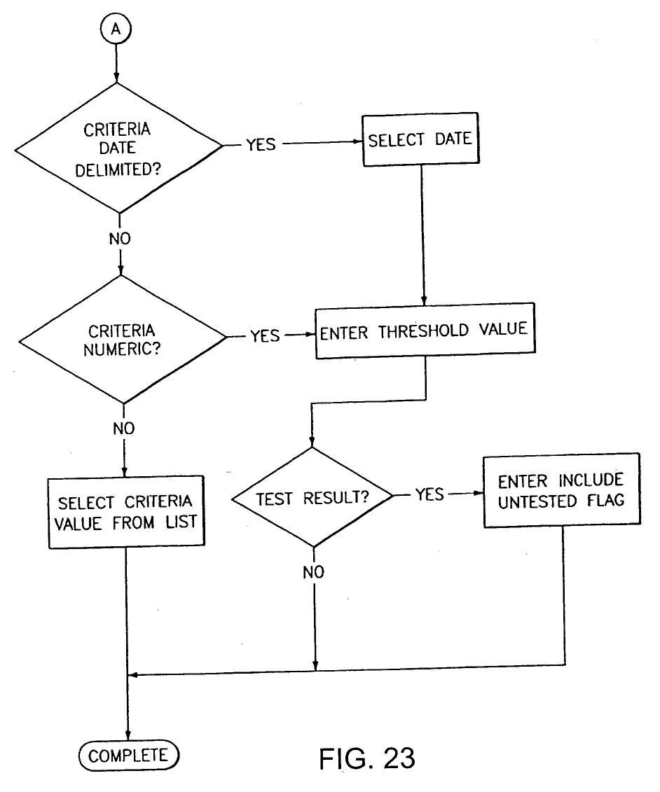

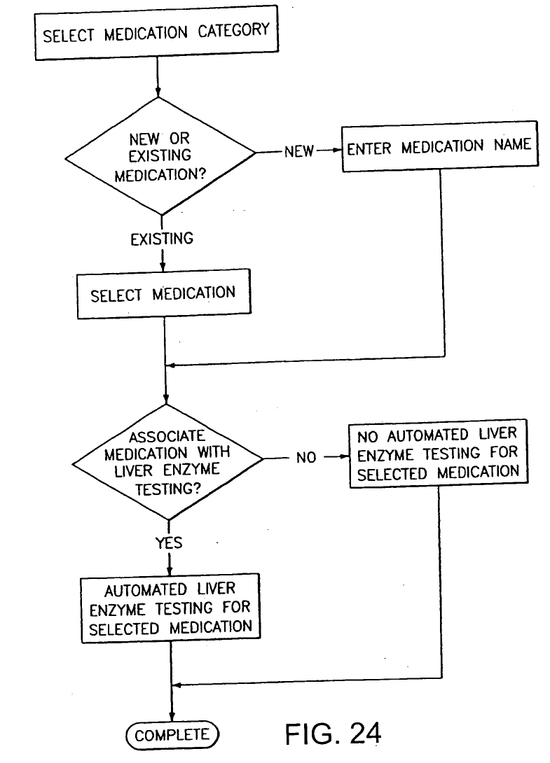

Now turning to FIGS. 22 and 23, the logic sequence for generation of high-risk monitor rule criteria is illustrated. The user enters the high-risk editor and may edit an existing rule or add a new rule (or criteria). The criteria includes patient parameters such as, HbAlc, lipid CDL), Microalbumin, Liver Enzyme (ALT), Liver Enzyme (AST), Complication, Comorbidity, Risk Factor, BMI, BP, ER Visits, Days Hospitalized, Days Lost From Activities, SMBG Daily Min, and SMBG Daily Max. Next, the user is prompted with respect to whether the criteria are date deliminated or numeric deliminated (and/or both). If a test result, patients who have not been tested may be included. Referring now to FIG. 24, the logic sequence to generate a high-risk rule for patients taking medication that may adversely impact the liver is illustrated. The FDA now recommends that liver enzyme testing be performed on patients taking specific medications. The user selects a medication category, is then prompted to determine if it is a new or existing medication, and is then prompted to determine whether the medication is associated with an adverse reaction. If the medication is associated with a liver reaction, a high-risk rule is generated to require testing for liver enzymes. If the enzyme is present, the action sequence is initiated (FIGS. 11A-11C).

In a further embodiment of the present invention, the chronic disease monitor 10 performs real-time monitoring, analysis, and reporting of patient outcomes, illustrating and substantiating the clinical effectiveness of medications, devices, and other chronic disease clinical and educational interventions. The automated process analyzes changes over time in software defined and stored clinical indicators. Outcomes are monitored, analyzed, and reported for single patients or patient populations. Patient data 16 can be aggregated from multiple locations, and standard or custom (both tabular and graphical) reports can be generated. Aggregated data is stripped of patient identifiers to protect confidentiality.

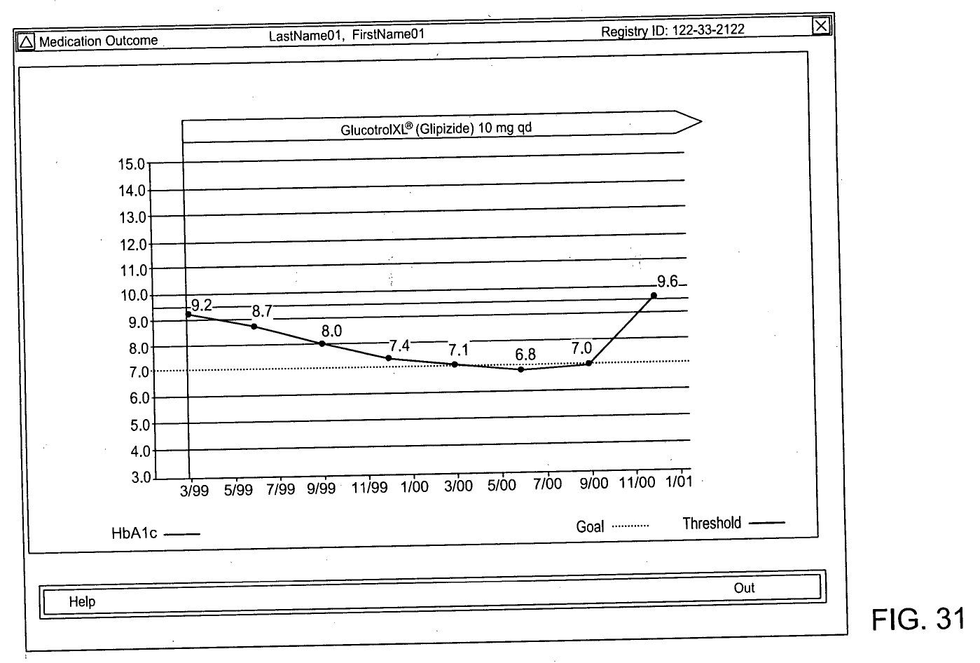

Moreover, the chronic disease monitor 10 enables tracking of educational intervention outcomes (for example, see FIG. 31). By tracking changes in patient clinical measures, the effectiveness of the educational intervention can be measured empirically. The chronic disease monitor 10 also automates the development and updating of individualized assessment and educational plans for each patient.

The chronic disease monitor 10 integrates comprehensive clinical management with the delivery and assessment of patient education, consistent with the National Standards for Diabetes Self-Management Education and other national standards.

The chronic disease monitor 10 produces automated, context-driven patient communications and education. It will produce multi-language correspondence that reminds patients about planned care events and offers results-based feedback about the most recent changes in their glycemic control. Digitally stored education materials are produced and scheduled for patient delivery based on a care plan customized for each patient.

In particular, the invention provides a chronic disease monitor 10 that incorporates the process steps of best practices, prevention and wellness programs, patient education, professional education, therapy goals and selection, integrated real-time quality assessment and reporting, utilization and compliance monitoring, and outcomes measurement and management. All of this information may be entered through the user interface 13 into the database 12 and stored therein according to an algorithm.

Moreover, the chronic disease monitor 10 is compatible with various standards bodies. For example, in a diabetes implementation, the invention is compatible with the American Diabetes Association (ADA), the ADA Standards of Care, and the National Standards for Diabetes Self-Management Education.

The chronic disease monitor 10, termed a “diabetes registry” in one possible implementation, supports physicians and other members of a diabetes care team by fully automating the planning, delivery, management, and ongoing quality assessment of patient care. The chronic disease monitor 10 provides software that conducts real-time monitoring of each patient's care, integrates and measures the results of patient education, and provides an ongoing systematic assessment of clinical outcomes. The chronic disease monitor 10 has the potential to advance the quality of diabetes care by giving physicians and other diabetes care team members the critical information and tools they need more effectively to help patients.

The chronic disease monitor 10 greatly assists a user in improving the quality of care by providing a program that gives up-to-date and complete information needed to help a patient manage his or her diabetes, and by producing an outcomes analysis that substantiates the value of interventions.

The chronic disease monitor 10 provides software algorithms that automate the creation and implementation of standards-based guidelines, and enable a diabetes clinical team to select the measures, parameters and thresholds that are used to collect, monitor and report the clinical status of all patients. The program assesses the status, measures the progress, and reports the results for all patients under care. The program assures the delivery of standards-based, cost-efficient, quality diabetes care—delivered to the appropriate patient at the appropriate time.

Furthermore, the chronic disease monitor 10 analyzes clinical results on an ongoing basis, producing alerts for results above established thresholds, reminders for scheduled tests and visits, and context-sensitive patient communication and education. The software includes a high-risk monitor that is active in the background of the program—building an ongoing high-risk patient assessment based on parameters set by the clinical team. This ongoing high-risk assessment creates an opportunity for earlier identification and intervention before the occurrence of high-expense care episodes.

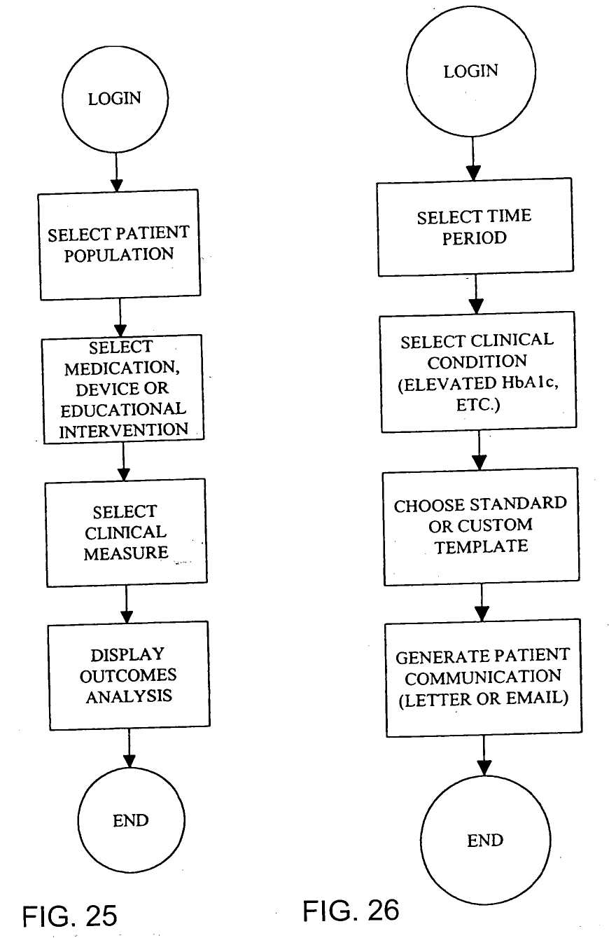

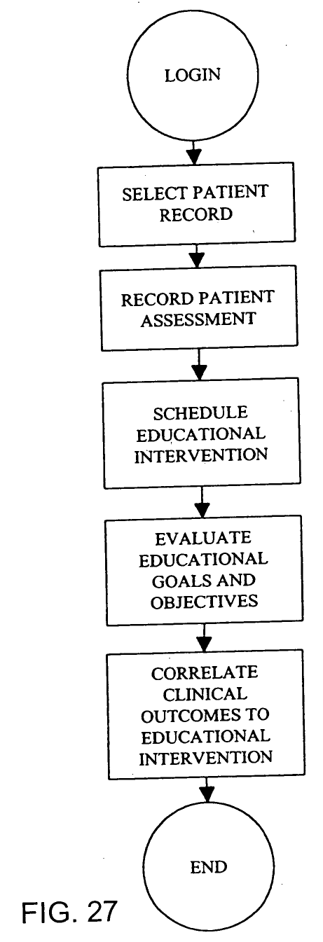

FIG. 25 is a block diagram representing the logic sequence for a patient outcome analysis performed by the system 10. A user, such as a physician, logs into the system 10 and, using an appropriate graphical window, discussed below, selects the desired patient population. The user then selects the medication, device or educational intervention whose effects are desired to be analyzed, and the clinical measure desired. The system 10 then displays the selected outcome on the screen of the terminal 18, either as a graph or as a chart, or on the printer 28. Program flow then terminates.

FIG. 26 is a block diagram representing the logic sequence for patient communications performed by the system 10. A user, such as a physician, logs into the system 10 and, using an appropriate graphical window, discussed below, selects the desired time period. The user then selects the desired clinical condition, which may be, for example, elevated HbAlc. The user then selects whether to employ a standard template or a custom template for the patient communication. The system 10 then generates the desired patient communication, which may be an e-mail 27 or a conventionally mailed letter printed on the printer 28. Program flow then terminates.

FIG. 26 is a block diagram representing the logic sequence for patient education performed by the system 10. A user logs into the system 10 and, using an appropriate graphical window, discussed below, selects a patient record. The user then records the patient assessment, and schedules an appropriate educational intervention. The patient's educational goals and objectives are then evaluated, and the system 10 then correlates the clinical outcomes to the educational intervention. Program flow then terminates.

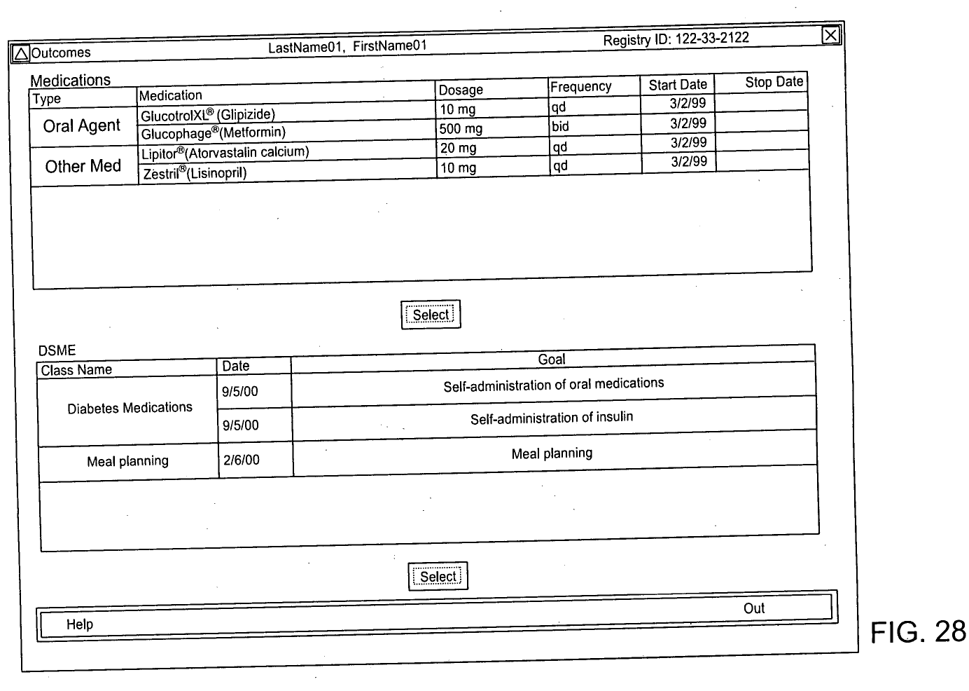

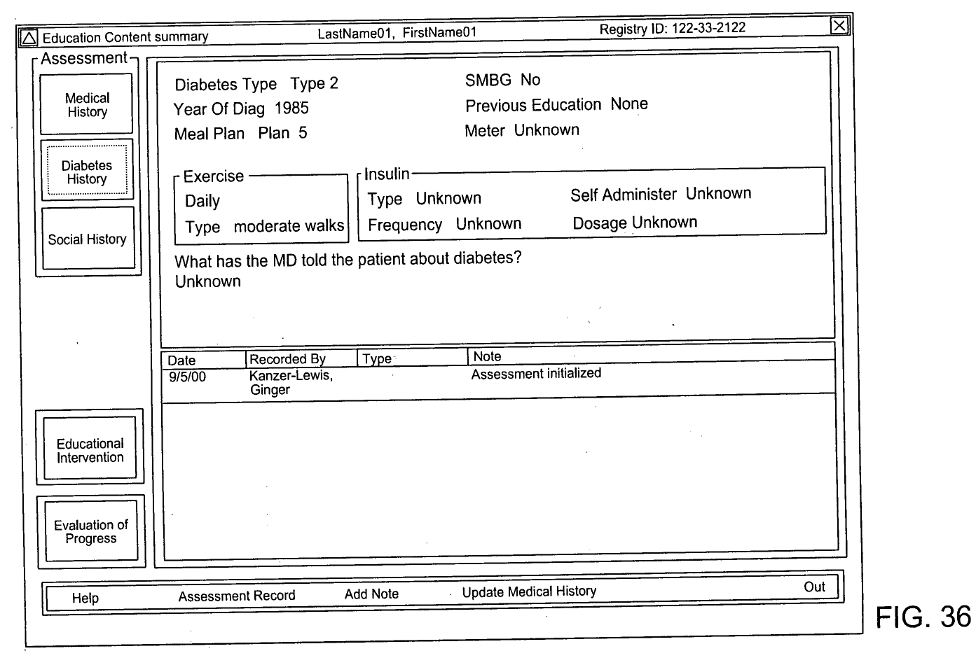

FIGS. 28-50 illustrate sample screen printouts that may be used to implement the invention in one possible further embodiment. It will be appreciated that, in FIGS. 28-50, the notation “LastNamexx, FirstNamexx” denotes a patient identifier.

FIG. 28 illustrates an “outcomes” screen that provides an overview of the medications and educational treatment plans for the patient. The screen of FIG. 28 provides, at the top, an indication of the medications that the particular patient has been taking. The name of the medication, dosage, frequency, start, and stop dates are indicated. These fields can be entered by the user using an appropriate interface device such as the terminal 18. Generally, the user of the chronic disease monitor 10 will be a doctor, doctor's office administrator, managed care administrator, or health care educator. The screen of FIG. 28 also indicates the names of classes that the patient is scheduled to attend. These educational courses are an integral part of the patient's treatment, and may be offered at any location, such as a hospital, doctor's office or school, or even via video tape, Internet and so forth. The patient may learn various skills in a class, such as how to take his or her medication, the important of proper nutrition and exercise, and so forth. The class name is indicated as well as the goals of the classes.

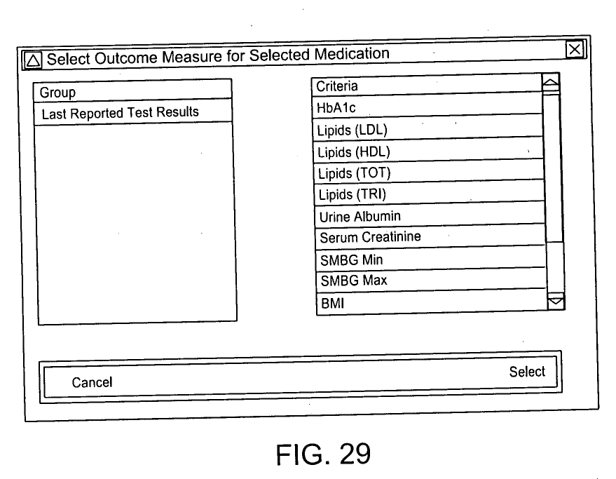

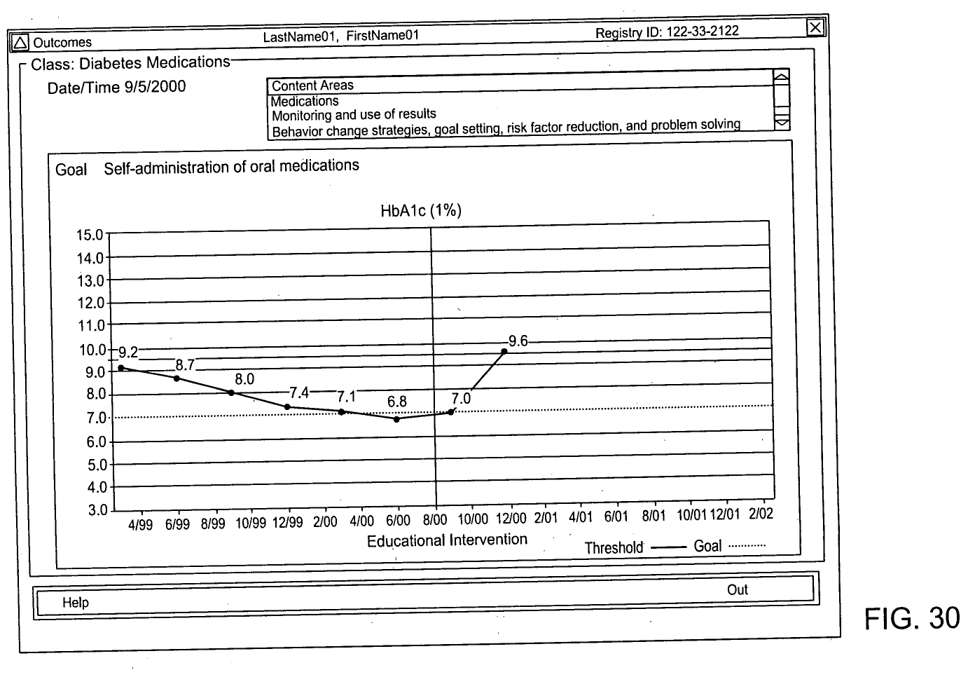

FIG. 29 illustrates how the user may select clinical measure criteria for the patient. The values for the particular measures may be input over time by the administrator based on test results. As an example, assume the desired measure to view is HbAlc. This measure is seen plotted versus time in FIG. 30. A benchmark is also shown that indicates when the patient attended a class (i.e., “educational intervention”). This allows the user to determine the trend of the measure, as well as the effects of the educational intervention. In the illustration of FIG. 30, it is seen that the educational intervention has a positive effect. This provides an important confirmation of the value of the education. Additional threshold and goal levels may be plotted as shown.

FIG. 31 shows an example of a medication outcome for HbAlc for the medication “GlucotrolXL®.”

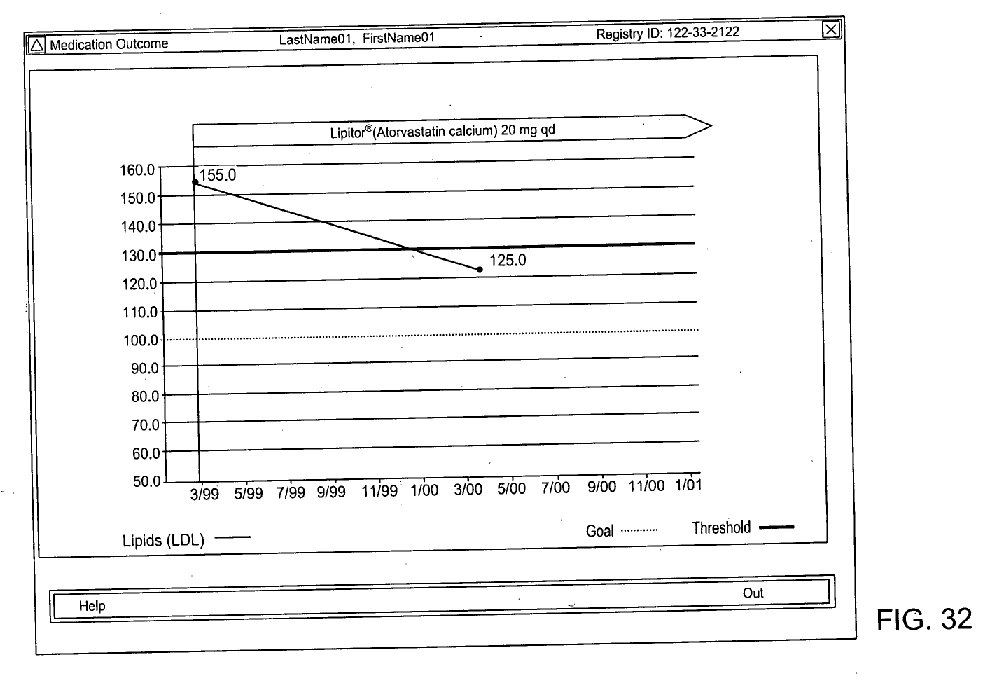

FIG. 32 shows an example of a medication outcome for LDL for the medication “Lipitor®”.

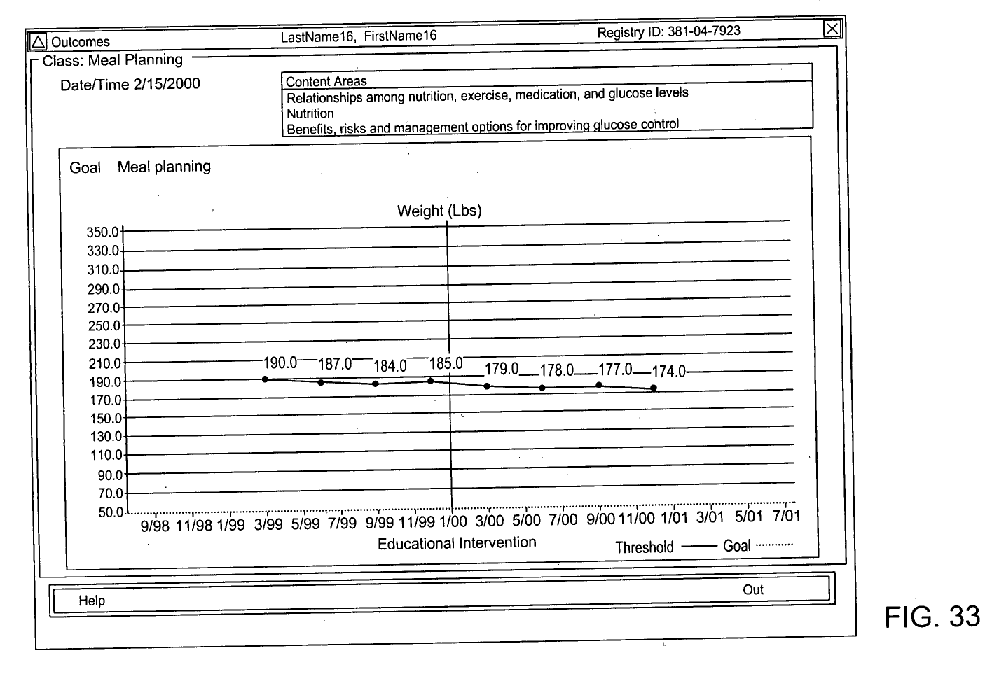

FIG. 33 shows an example of an outcome for patient weight, plotted versus time. Additionally, the educational intervention is shown.

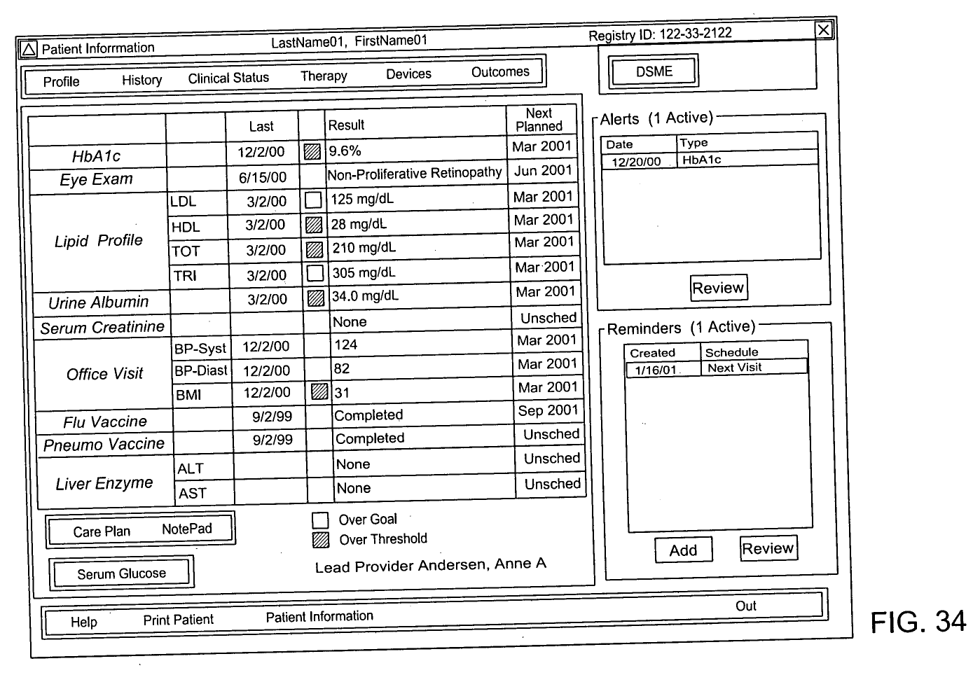

FIG. 34 illustrates a patient information screen. Here, the user may scroll down to view specific information, such as alerts and reminders, for the various factors shown. For example, the factor “Lipid Profile” is selected, as indicated by the highlight. A corresponding alert is that an HbAlc measure is to be taken on a certain date. A corresponding reminder is that the patient's next doctor's visit is scheduled for a certain date. Additionally, a history and results of the various factors are shown, as well as a color-coded highlight for results that are out of range.

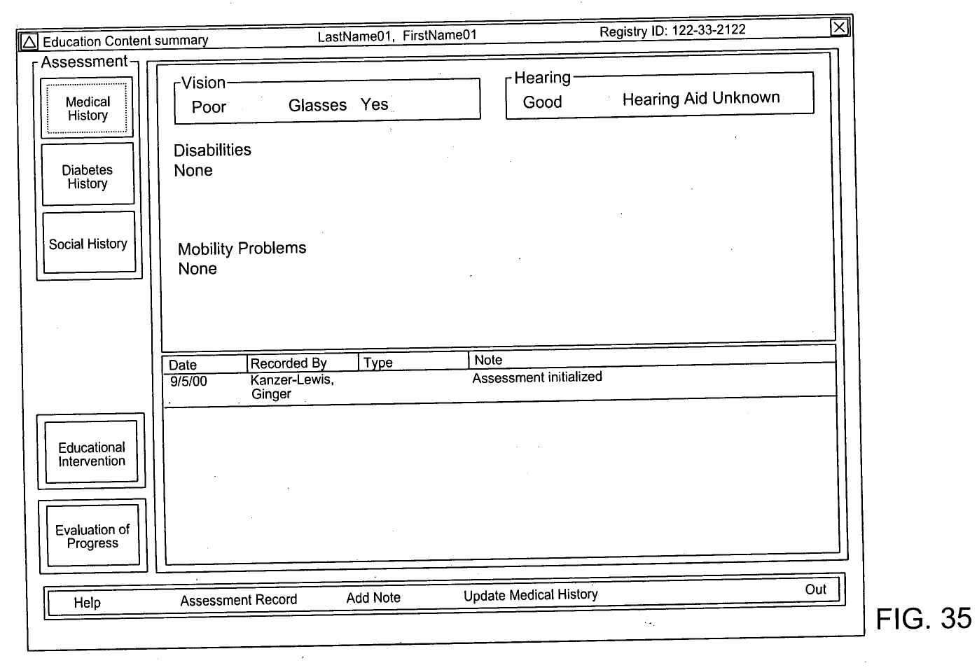

FIG. 35 illustrates an education content summary—medical history screen. Note that various assessment factors may be selected by the user, such as medical history, diabetes history, social history, educational intervention, and evaluation of progress. For example, with medical history selected, information is entered regarding the patient's vision, hearing, and so forth. Moreover, the system documents who made the entry and when, and any other relevant notes. FIG. 35 illustrates a specific application of an education content summary, i.e., for a patient's diabetes history screen. Here, information such as the patient's diabetes type, year of diagnosis and so forth are entered by the user.

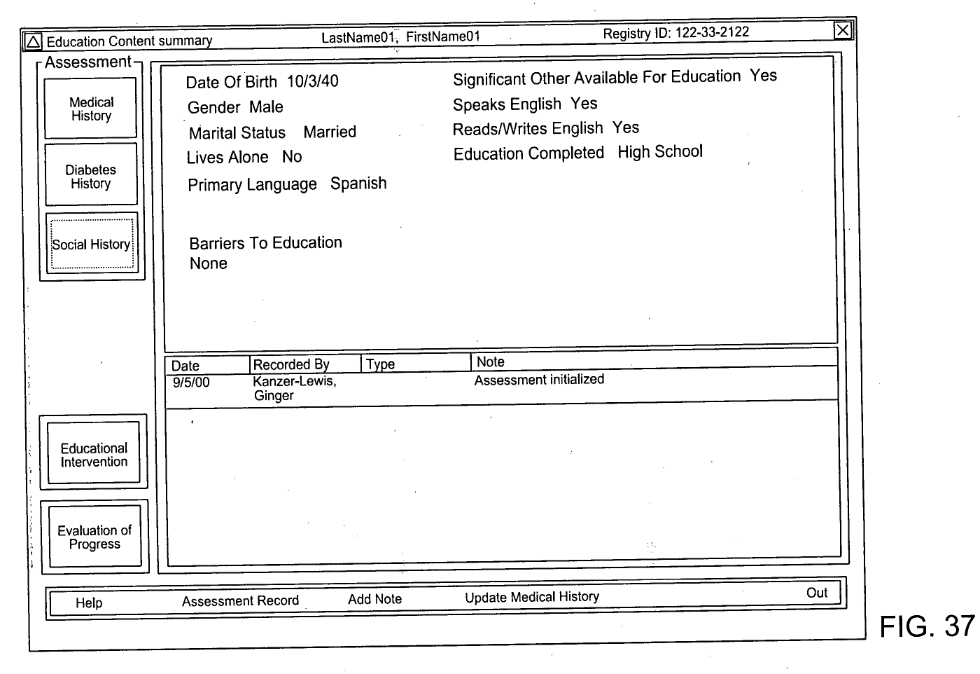

FIG. 37 illustrates an education content summary—social history screen. Here, information such as the patient's date of birth, marital status, and so forth are entered.

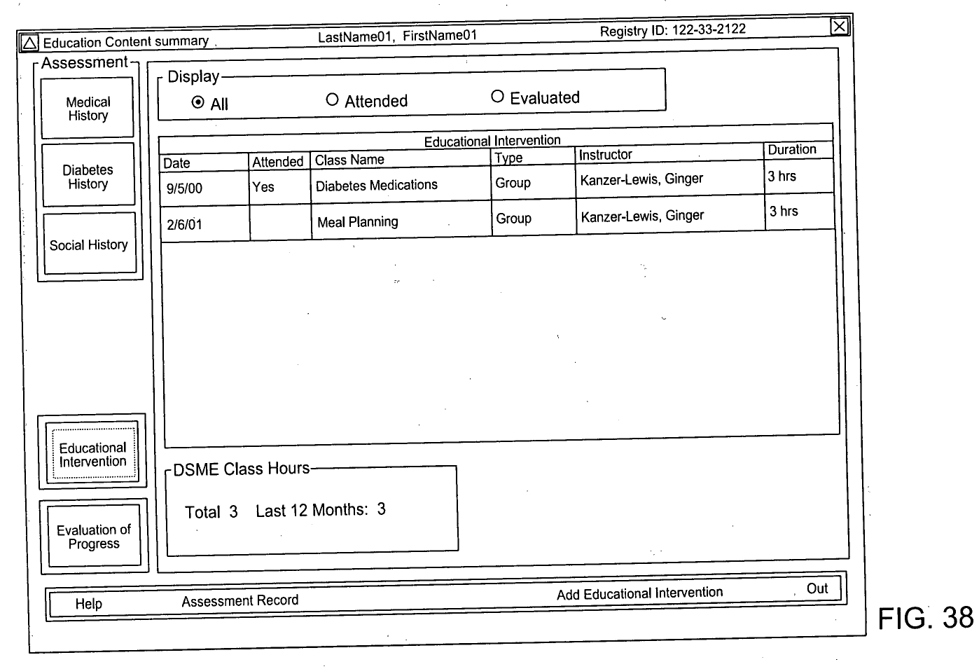

FIG. 38 illustrates an education content summary—educational intervention. Here, information is entered regarding educational courses that the patient has taken, or is assigned to take.

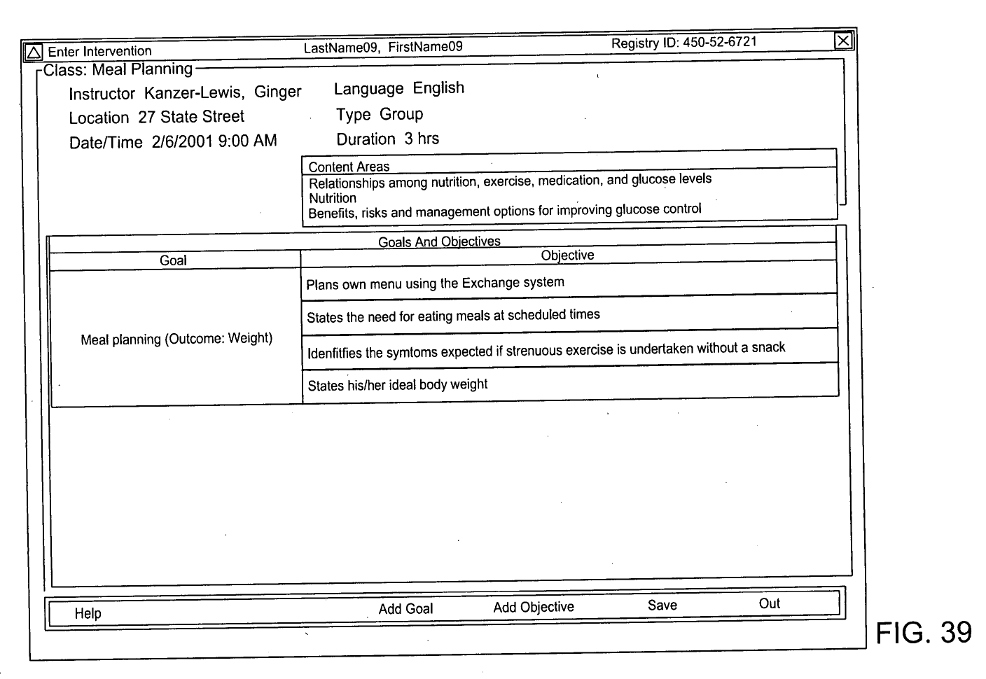

FIG. 39 illustrates an enter intervention screen. This interface allows the user to view details of a particular class. For example, for the class “Meal Planning” (see also FIG. 38), detailed information is provided at FIG. 39 regarding the objectives of the class. This allows the user to determine if the class is appropriate for the patient, and provides administrative information such as the location of the class and the instructor's name.

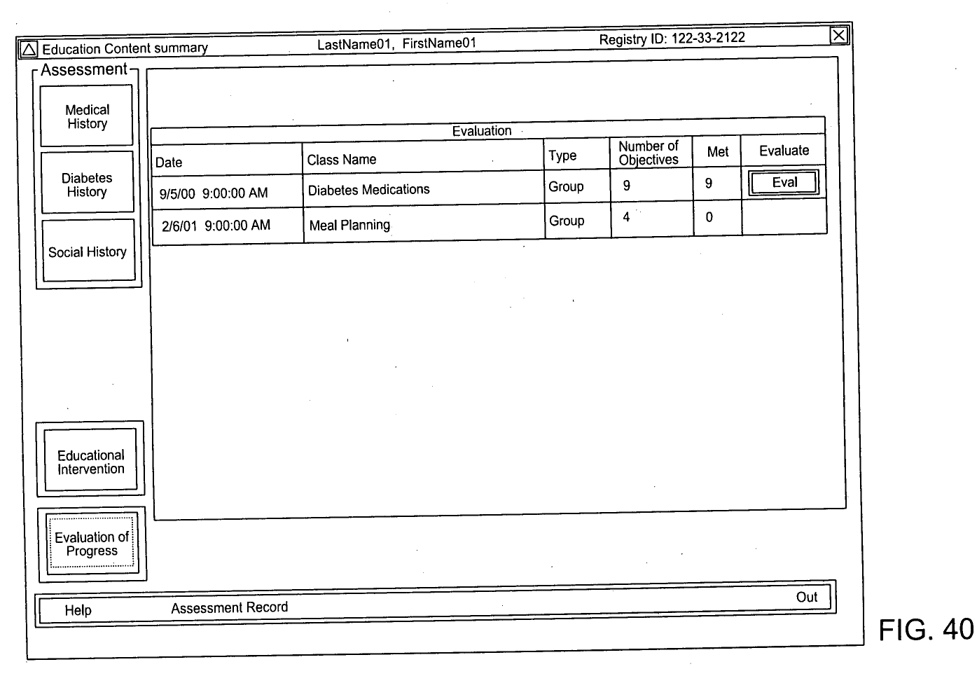

FIG. 40 illustrates an education content summary—evaluation of progress screen. Here, the user can enter information regarding how many of the class objectives have been met by the patient.

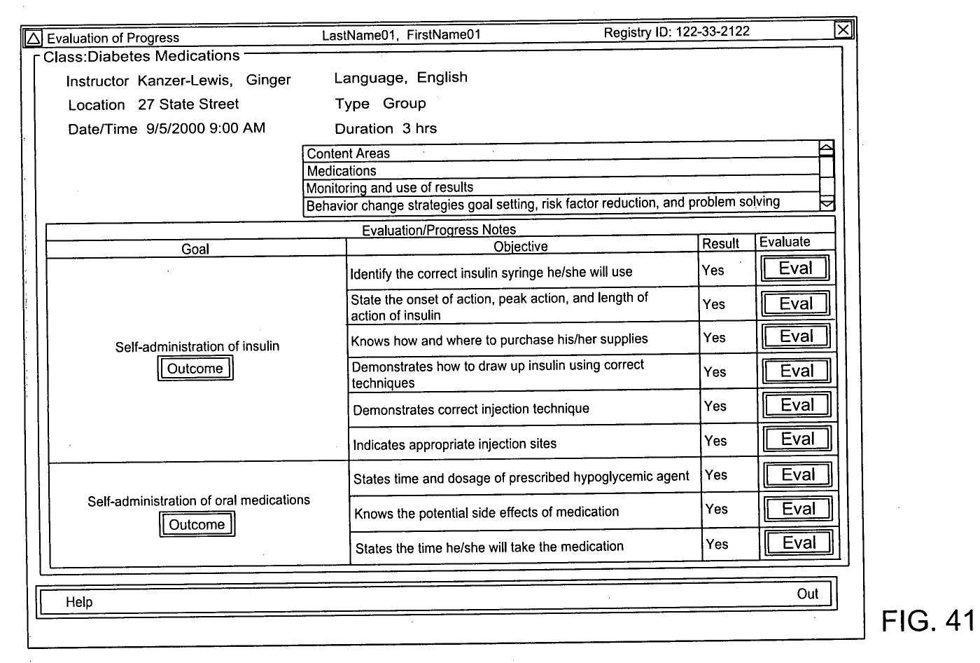

FIG. 41 illustrates further details of the evaluation of progress function, e.g., to allow the user to view each objective and indicate whether it has been met.

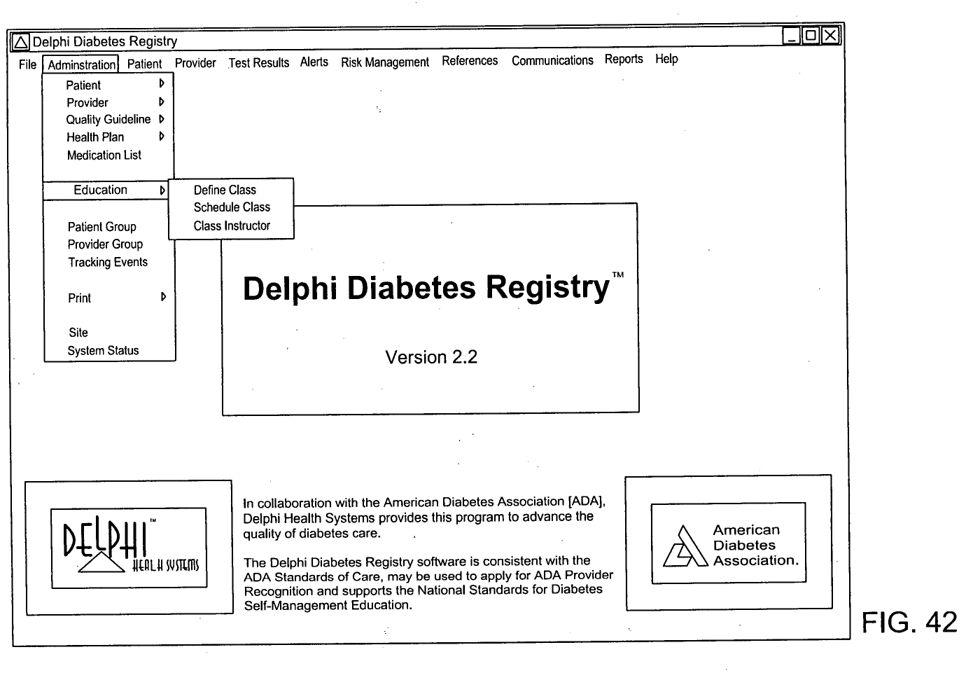

FIG. 42 illustrates a “diabetes registry” that provides a variety of functions. For example, one function regarding education allows the user to define a class, schedule a class, or indicate a class instructor.

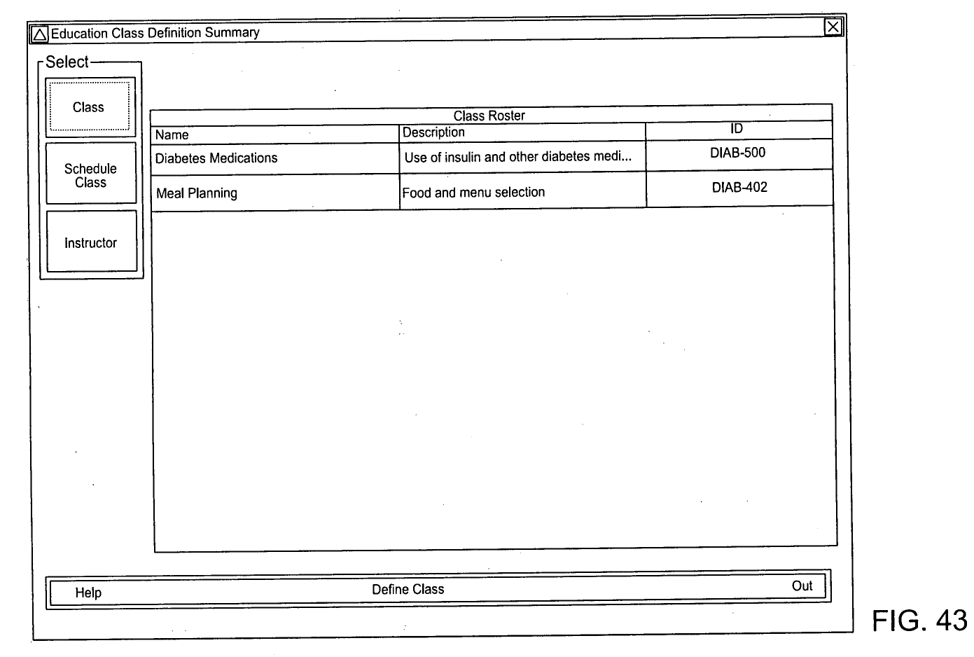

FIG. 43 illustrates an education class definition summary that provides functions to allow the user to enter and view information. A class function indicates, e.g., the name of the class, its description, and its identifier number.

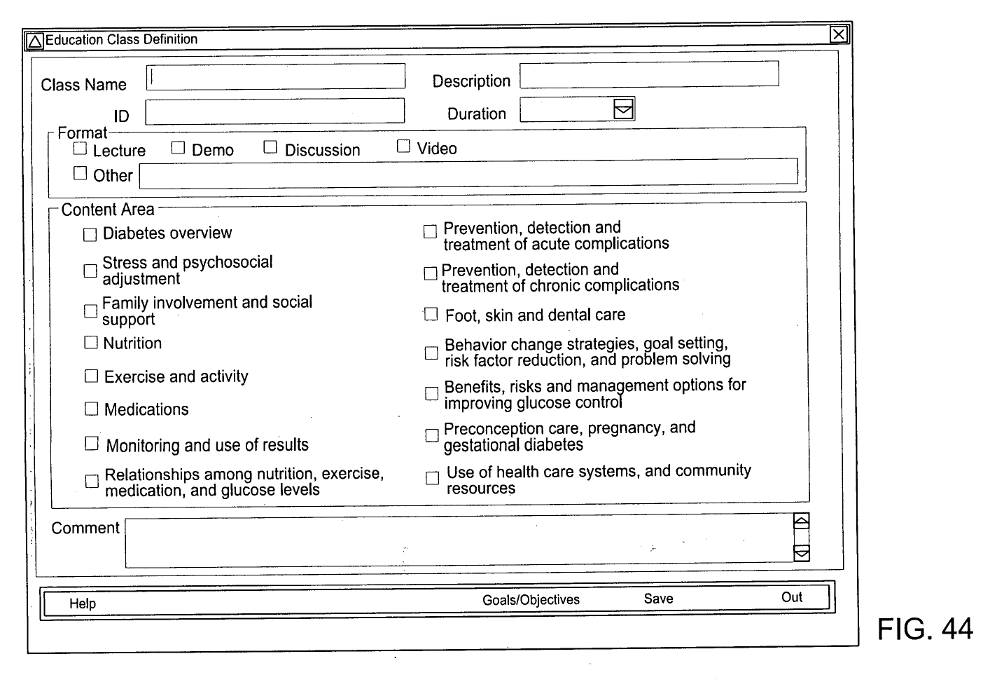

FIG. 44 illustrates an education class definition screen that allows the user to enter information regarding a class, such as its name and description. Moreover, the user denotes whether the class provides information that is related to various standardized content areas.

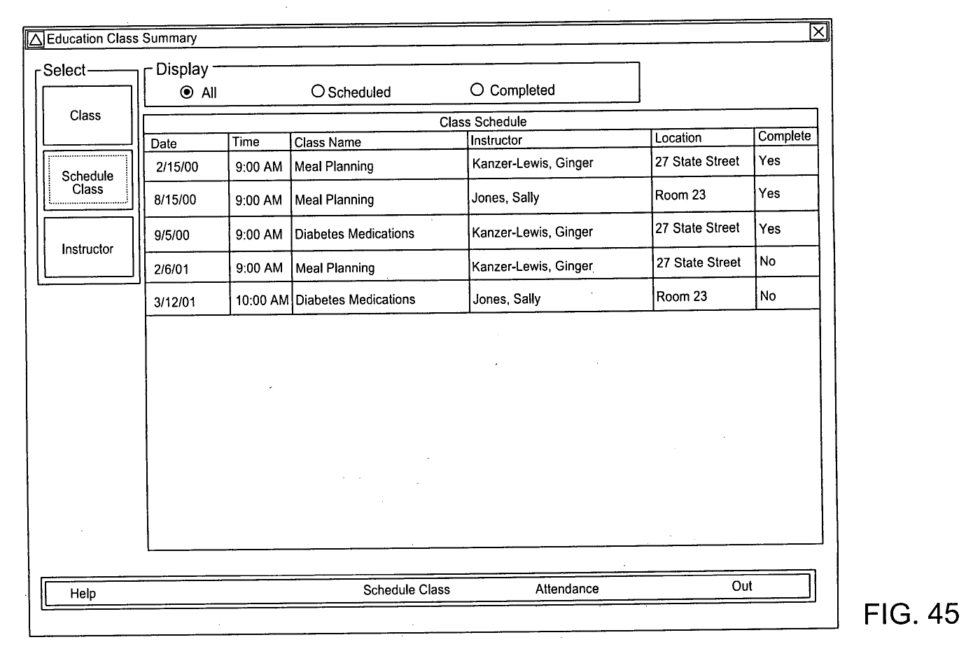

FIG. 45 illustrates an education class summary schedule class screen that allows the user to review class-scheduling information.

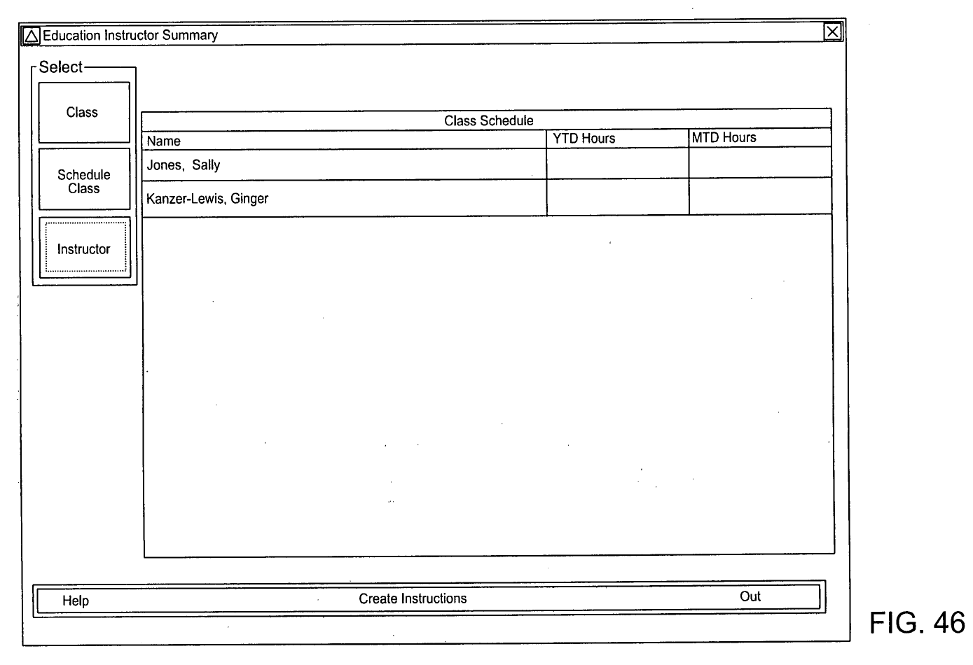

FIG. 46 illustrates an education class summary—instructor screen that provides summary information regarding the various class instructors.

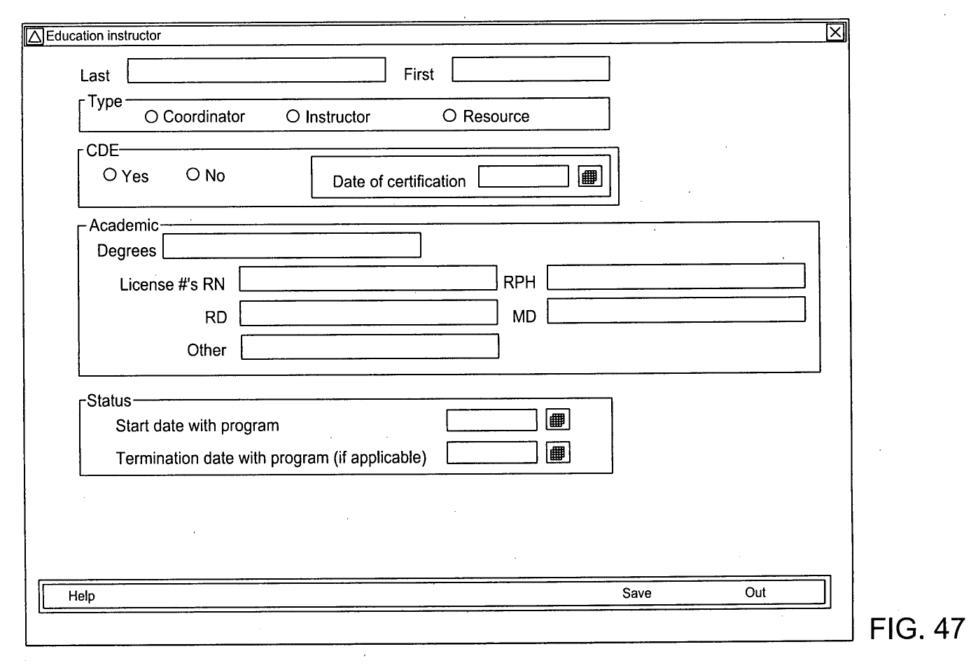

FIG. 47 provides a data entry screen for use in connection with the screen of FIG. 44. It should be appreciated that documentation of the credentials of the instructors may be important for compliance with some standards groups.

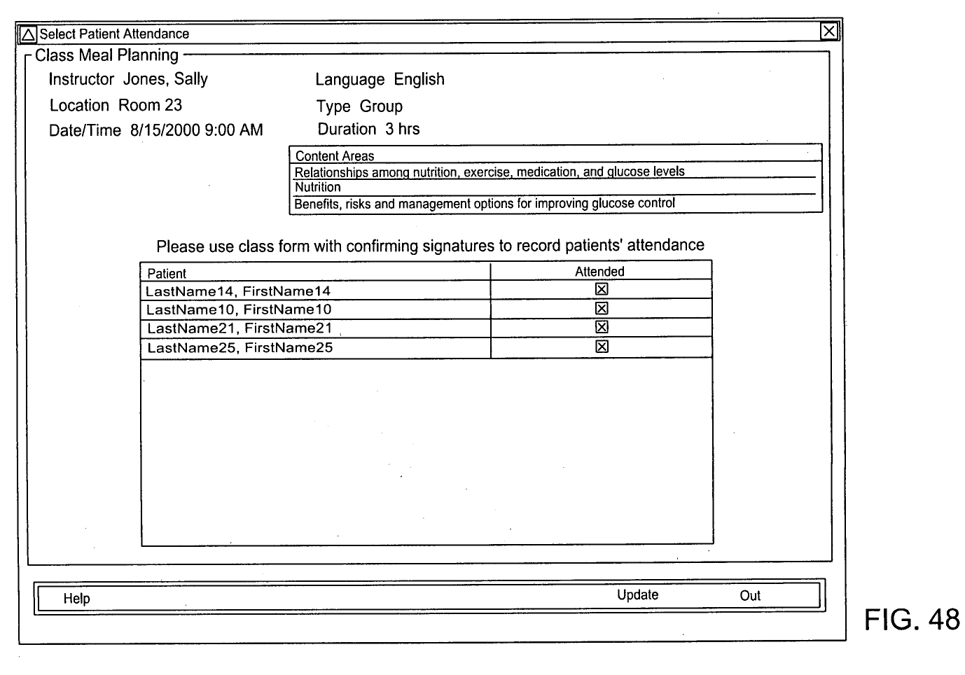

FIG. 48 provides a select patient attendance screen that allows a user, such as a class instructor, to enter attendance information for patients.

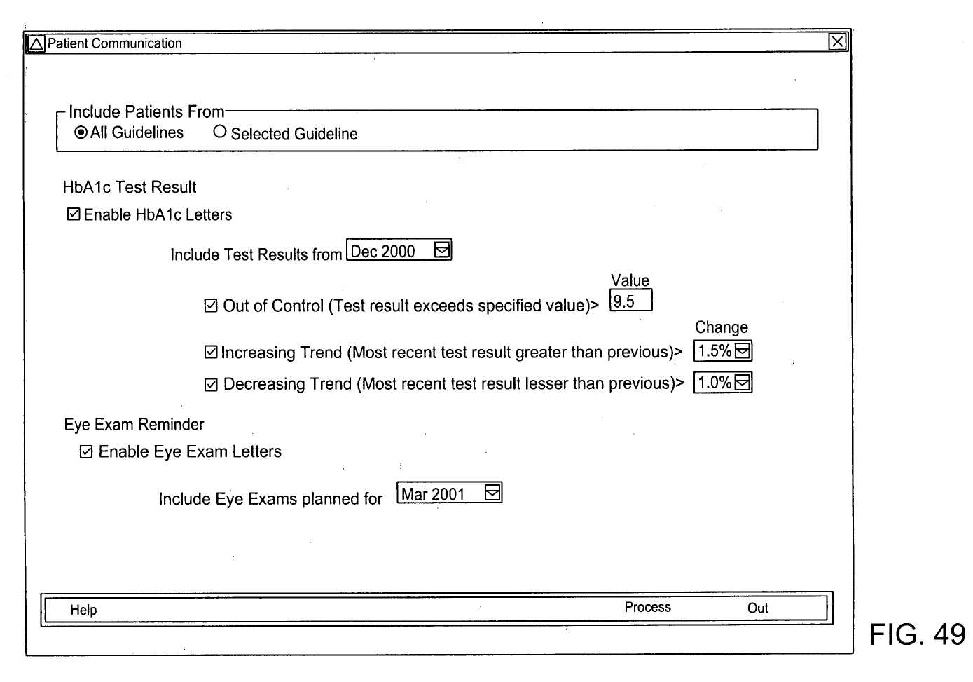

FIG. 49 illustrates a patient communication screen that allows a user to initiate communications, such as printed letters or e-mails 27, to patients based on selected criteria. The communications may be reminders for upcoming appointments, or notifications that recent test results are out of range, or are on an increasing or decreasing trend. This is a great time saver, as the user can contact selected patients from a large population to receive a communication. These ongoing communications are important for managing chronic diseases such as diabetes to keep the patient informed of his or her medical condition, and provide a convenience in reminding him or her of important upcoming events.

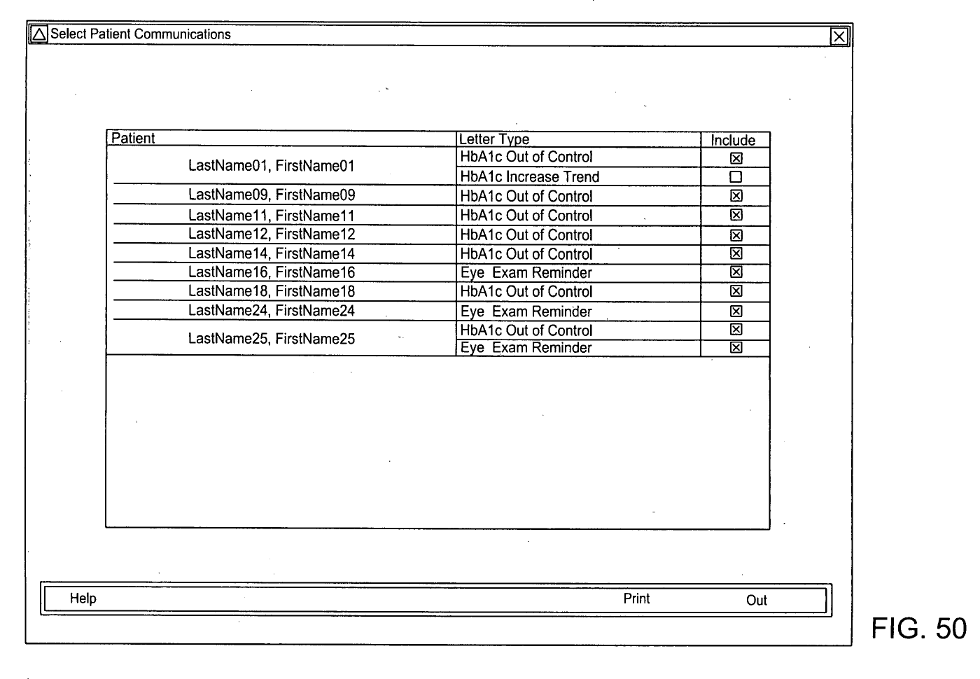

FIG. 50 illustrates a select patient communications screen that lists selected patients based on the selection criteria input by the user via the screen of FIG. 49. This allows the user to identify the patients that fall under the criteria and individually to select or de-select them from receiving a communication. Additionally, the particular type of communication is indicated. Accordingly, it can be seen that the chronic disease monitor 10 provides a chronic disease monitoring, education and patient communication system that provides a variety of features, including setting patient outcomes, providing education, and automating patient communications.

The chronic disease monitor 10 allows the user to monitor and manage patients, as well as generating a care plan. The chronic disease monitor 10 provides conformance with industry and governmental guidelines for patient education. The chronic disease monitor 10 allows users to use their professional judgment and assessment results such as test results to create educational programs tailored to the patient, including the creation of new types of classes with specific objectives. The chronic disease monitor 10 facilitates the monitoring of a large number of patients, and allows aggregation of data. This is particularly important, e.g., to pharmaceutical companies that wish to obtain concrete evidence of the effectiveness of their medications. The chronic disease monitor 10 also provides proof of effectiveness for the program educators, care providers and other.

Although preferred embodiments of the present invention have been described in detail herein with reference to the accompanying drawings, it is to be understood that the invention is not limited to those precise embodiments, and that various changes and modifications may be effected therein by one skilled in the art without departing from the spirit and scope of the invention as defined in the appended claims.

1. A chronic disease outcomes, communication, and education system comprising:
a. a database for storing a plurality of patient data entries, each of said patient data entries including personal information of a patient and a guideline concerning the patient's care, said guideline including a default test value associated with monitoring a chronic disease;
b. a user interface for entering patient entries for storage in said database and queries into said database related to patient monitoring, communication and education; and
c. a processor for retrieving said patient data entries from said database in response to said queries from said user interface and storing said patient data entries in said database;
d. wherein said processor provides patient and population outcomes analysis according to an algorithm.

2. The system of claim 1, wherein said processor generates reports of clinical outcomes of at least one of medications, medical devices, educational interventions, and chronic disease interventions in response to said patient entries and said algorithm.

3. The system of claim 2, wherein said program generates said reports of clinical outcomes for a selected patient.

4. The system of claim 2, wherein said program generates said reports of clinical outcomes for a population.

5. The system of claim 1, wherein said program analyzes clinical outcomes of at least one of medications, medical devices, educational interventions, and chronic disease interventions in response to said patient entries and said algorithm.

6. The system of claim 5, wherein said program analyzes said clinical outcomes for a selected patient.

7. The system of claim 5, wherein said program analyzes said clinical outcomes for a population.

8. The system of claim 1, wherein said program monitors clinical outcomes of at least one of medications, medical devices, educational interventions, and chronic disease interventions in response to said patient entries and said algorithm.

9. The system of claim 8, wherein said program monitors said clinical outcomes for a selected patient.

10. The system of claim 8, wherein said program monitors said clinical outcomes for a population.

11. The system of claim 1, wherein said program generates a care plan customized for each patient based upon said patient data and said guideline for said patient's care entered into said database.

12. The system of claim 11, wherein said processor generates said care plan based upon standards as set by standards bodies entered into said database.

13. The system of claim 1, wherein said program generates notification messages to at least one of said patient, a physician, a health care plan administrator, and a health care provider.

14. The system of claim 1, wherein said database stores information regarding patient educational programs, and wherein said user enters said information regarding patient educational programs from said user interface.

15. The system of claim 14, wherein said information regarding patient educational programs includes at least one of instructor name, instructor qualifications, and class schedules.

16. The system of claim 1, wherein said user interface stores a record of educational programs attended by said patient in said database, and wherein said processor measures a condition of said patient, selectively compares said measured condition to a treatment monitoring threshold and said record of educational programs attended by said patient, and generates an outcomes analysis according to said algorithm in response thereto.

17. The system of claim 1, wherein said user interface stores high-risk patient assessment parameters in said database, and said processor includes a high-risk monitor responsive to said high-risk patient assessment parameters for identifying patients at high-risk.

18. The system of claim 17, wherein said monitor is a diabetes registry that automates the planning, delivery, management, and ongoing quality assessment of said care of said patient.

19. The system of claim 1, wherein said processor generates notification messages to at least one of said patient, a physician, a health care plan administrator, and a health care provider.

20. The system of claim 19, wherein said processor generates said notification messages as reminders for at least one of scheduled tests, planned educational programs, scheduled office visits, scheduled treatment steps, measured conditions, missed treatment steps, and a trend in measured conditions.

21. The system of claim 19, wherein said notification messages are written in one of a plurality of languages.

22. The system of claim 19, wherein said user interface stores a treatment guideline defining at least one treatment and a predetermined treatment monitoring threshold for said treatment in said database; and wherein said processor measures a condition of said patient in accordance with said treatment, selectively compares said measured condition to said treatment monitoring threshold, and generates said notification message when said measured condition exceeds said predetermined treatment monitoring threshold.

23. The system of claim 1, wherein said reports are displayed in a standard template.

24. The system of claim 23, wherein said reports are at least one of graphical representations and tabular representations.

25. The system of claim 1, wherein said reports are displayed according to a template customized by a user.

26. The system of claim 25, wherein said reports are at least one of graphical representations and tabular representations.

27. The system of claim 1, and further comprising an off-site user interface that communicates with said processor.

28. In a chronic disease outcomes, communication, and education system comprising a database for storing a plurality of patient data entries, each of said patient data entries including personal information of a patient and a guideline concerning the patient's care, said guideline including a default test value associated with monitoring a chronic disease, a user interface for entering patient entries for storage in said database and queries into said database related to patient monitoring, communication and education, and a processor for retrieving said patient data entries from said database in response to said queries from said user interface and storing said patient data entries in said database, the improvement comprising a computer program embodied on a computer-readable medium for operating said processor to provide patient and population outcomes analysis according to an algorithm.

29. The improvement of claim 28, wherein said program generates reports of clinical outcomes of at least one of medications, medical devices, educational interventions, and chronic disease interventions in response to said patient entries and said algorithm.

30. The improvement of claim 29, wherein said program generates said reports of clinical outcomes for a selected patient.

31. The improvement of claim 29, wherein said program generates said reports of clinical outcomes for a population.

32. The improvement of claim 29, wherein said program analyzes clinical outcomes of at least one of medications, medical devices, educational interventions, and chronic disease interventions in response to said patient entries and said algorithm.

33. The improvement of claim 32, wherein said program analyzes said clinical outcomes for a selected patient.

34. The improvement of claim 32, wherein said program analyzes said clinical outcomes for a population.

35. The improvement of claim 29, wherein said program monitors clinical outcomes of at least one of medications, medical devices, educational interventions, and chronic disease interventions in response to said patient entries and said algorithm.

36. The improvement of claim 35, wherein said program monitors said clinical outcomes for a selected patient.

37. The improvement of claim 35, wherein said program monitors said clinical outcomes for a population.

38. The improvement of claim 28, wherein said program generates a care plan customized for each patient based upon said patient data and said guideline for said patient's care entered into said database.

39. The improvement of claim 28, wherein said program generates notification messages to at least one of said patient, a physician, a health care plan administrator, and a health care provider.

40. The improvement of claim 39, wherein said notification messages are messages as reminders for at least one of scheduled tests, planned educational programs, scheduled office visits, scheduled treatment steps, measured conditions, missed treatment steps, and a trend in measured conditions.

41. A computer program embodied on a computer-readable medium for operating a computer system including a database for storing a plurality of patient data entries, each of said patient data entries including personal information of a patient and a guideline concerning the patient's care, said guideline including a default test value associated with monitoring a chronic disease, a user interface for entering patient entries for storage in said database and queries into said database related to patient monitoring, communication and education, and a processor for retrieving said patient data entries from said database in response to said queries from said user interface and storing said patient data entries in said database, comprising a subroutine for operating said processor to provide patient and population outcomes analysis according to an algorithm.

42. The computer program of claim 41, wherein said processor generates reports of clinical outcomes of at least one of medications, medical devices, educational interventions, and chronic disease interventions in response to said patient entries and said algorithm.

43. The computer program of claim 42, wherein said program generates said reports of clinical outcomes for a selected patient.

44. The computer program of claim 42, wherein said program generates said reports of clinical outcomes for a population.

45. In a chronic disease outcomes, communication, and education system including a database for storing a plurality of patient data entries, each of said patient data entries including personal information of a patient and a guideline concerning the patient's care, said guideline including a default test value associated with monitoring a chronic disease, a user interface for entering patient entries for storage in said database and queries into said database related to patient monitoring, communication and education, and a processor for retrieving said patient data entries from said database in response to said queries from said user interface and storing said patient data entries in said database, the improvement comprising a computer data signal embodied in a carrier wave representing sequences of instructions which, when executed by said processor, cause said processor to provide patient and population outcomes analysis according to an algorithm.

46. The improvement of claim 45, wherein said processor generates reports of clinical outcomes of at least one of medications, medical devices, educational interventions, and chronic disease interventions in response to said patient entries and said algorithm.

47. The improvement of claim 46, wherein said program generates said reports of clinical outcomes for a selected patient.

48. The improvement of claim 46, wherein said program generates said reports of clinical outcomes for a population.

49. A method of operating a computer processing system to provide chronic disease outcomes, communication, and education comprising the steps of:
a. storing a plurality of patient data entries, each of said patient data entries including personal information of a patient;
b. storing a treatment guideline defining at least one treatment and a predetermined treatment monitoring threshold for said treatment;
c. measuring a condition of said patient in accordance with said treatment;
d. selectively querying said database for factors related to patient and population outcomes analysis according to an algorithm; and
e. retrieving said patient data entries from said database in response to said queries from said user interface and storing said patient data entries in said database.

50. The system of claim 49, and further comprising the step of transmitting a notification message to at least one of the patient, a physician, a health care provider, and a health care plan administrator.
